(19) United States
(12) Patent Application Publication
(10) Pub. No.: US 2004/0044304 A1
(43) Pub. Date: Mar. 4, 2004

(54) Local drug delivery using photosensitizer-mediated and electromagnetic radiation enhanced vascular permeability

(76) Inventors: John S Hill, Solvang, CA (US); Jeffrey P Walker, Goleta, CA (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/181,463

(22) PCT Filed: Jan. 22, 2001
(86) PCT No.: PCT/US01/01981

(51) Int. Cl. A61K 31/555; A61N 1/30; A61K 9/00; A61K 31/409

(52) U.S. Cl. 604/20; 514/185; 514/410

(57) ABSTRACT

The invention relates to the site specific delivery of drugs in an organism. The described methods facilitate the delivery of a therapeutic or diagnostic drug by increasing vascular permeability in a site specific manner. Vascular permeability is enhanced in the disclosed methods by using a combination of a photosensitizer and radiation applied to a site of interest.

### FIELD OF THE INVENTION

The invention relates to the field of site specific drug delivery. The methods of the invention use a photosensitizer and radiation to enhance the permeability of biological tissue, especially blood vessels, to facilitate the delivery of a drug in a site specific manner.

### BACKGROUND OF THE INVENTION

Local Drug Delivery

The ability to deliver a drug to a localized area in a complex organism can be desirable. For example, many drugs show side effects that can be reduced or avoided if the drug is only delivered to a limited area in the organism. The delivery of diagnostic or therapeutic agents to specific sites in an organism presents a difficult challenge, especially in complex organisms like humans. Techniques that have been employed to deliver agents in a site specific manner are local injection of the agent, arterial or venous injection, and depot and/or slow release reservoirs designed to release the agent at a particular site.

Attempts to target drugs by using antibodies have not achieved site specificity. The problems using these techniques relate to, among other things, the typically unpredictable or extensive distribution of target epitopes (Dubowchik et al., 1999, Pharmacol. Ther. 83:67-123; Adams, 1998 In Vivo 12:11-21; Reilly et al., 1995, Clin. Pharmacokinet. 28:126-142; Klingermann et al., 1996, Mol. Med. Today 2:154-159; Verhoeyen et al., 1995, Biochem. Soc. Trans. 23:1067-1073).

Other attempts to deliver agents to the specific site have used vasoactive compounds to increase the permeability of blood vessels and thereby facilitate the uptake of the drug. However, these methods cannot deliver a drug to a locally confined site because the vasoactive compounds cannot be locally confined, leading to increased drug uptake in extended areas throughout the organism (Koga et al., 1999, J. Neurooncol. 43:153-151; Barnett et al., 1999, Cancer Gene Ther. 6:14-20; Barth et al., 1999, Neurosurgery 44:351-359; Cloughesy et al., 1999, Neurosurgery 44:270-279; Rainov et al., 1999, Hum. Gene Ther. 10:311-318; Jolliet-Riant et al, 1999, Fundam. Clin. Pharmacol. 13:16-26; Ford et al., 1998, Eur. J. Cancer 34:1807-1811; Kroll et al., 1998, Neurosurgery 43:879-889; LeMay et al., 1998, Hum. Gene Ther. 9:989-995; Fike et al., 1998, Neurooncol. 37:199-215; Kroll et al., 1998, Neurosurgery 42:1083-1099; Sugita et al., 1998, Cancer Res. 5i:914-920; Matsukado et al., 1997, J. Neurooncol. 34:131-138; Black et al., 1997, J. Neurosurg. 86:603-609; Bartus et al., 1996, Exp. Neurol. 142:14-28; Elliott et al., 1996, Exp. Neurol. 141:214-224; Matsukado et al., 1996, Neurosurgery 39:125-133; Koga et al., 1996, Neurol. Res. 18:244-247; Elliott et al., 1995, Invest. Ophthahnol. Vis. Sci. 36:2542-2547).

Thus, a need exists for methods to supply drugs to specific sites in complex organisms. The present invention provides such methods. By selectively increasing the permeability of a desired target tissue in an organism, the methods of the invention facilitate the delivery of a drug to that target tissue. The disclosed methods employ a targeted modulation of tissue properties. Tissue targeting techniques have been employed in photodynamic therapy, although such techniques are designed for the destruction of hyperproliferating and abnormal tissue.

Photodynamic Therapy

Photodynamic therapy (PDT) is a therapeutic procedure designed for the destruction of pathological tissues in a patient, for example, cancer tissue or blood vessels during hypervascularization. In PDT, a photosensitizing agent is delivered to the pathological tissue and radiation is applied to destroy that tissue. For example, when tumors undergo PDT, the photosensitizing agent is delivered to the patient, the agent is then allowed to distribute throughout the cancerous tissue, which is then exposed to radiation. The radiation of the photosensitizing agent in the tissue leads to, for example, the generation of radicals and, ultimately, the destruction of the cancerous tissue.

A biological effect of PDT is the targeted destruction of both cells and surrounding vasculature. It is believed that cells within the target field can be destroyed by both apoptotic (Godar, 1999, J. Investig. Dermatol. Symp. Proc. 4:17-23; Oleinick et al., 1998, Radiat Res. 150(5 Suppl):S146-56) and necrotic pathways (Oleinick et al., 1998, Radiat Res. 150(5 Suppl):S146-56). In addition, it has been shown that vasculature and microvasculature in tumors and normal tissues are shut down and destroyed in PDT. The exact mechanisms by which this vascular effect is mediated are unknown but appear to result in thrombosis and vascular stasis followed by vessel wall breakdown within 24 hours. The data in the literature suggests that the effects are threshold in nature, in other words, once a critical PDT threshold is reached, vascular destruction results (Wang et al., 1997, Br. J. Dermatol. 136:184-189; Liu et al., 1997, Cancer Lett. 111:157-165; Fingar, 1996, J. Clin. Laser. Med. Surg. 14:323-328; Brasseur et al., 1996, Photochem. Photobiol. 64:702-706; van Geel et al., 1996, Br. J. Cancer 73:288-293; Iliaki et al., 1996, Lasers. Surg. Med. 19:311-323; Schmidt-Erfurth et al., 1994, Ophthalmology. 101:1953-1961; McMahon et al., 1994, Cancer Res. 54:5374-5379; Tsilimbaris et al., 1994, Lasers. Surg. Med. 15:19-31; Fingar et al., 1993, Photochem. Photobiol. 58:393-399; Fingar et al., 1993, Photochem. Photobiol. 58:251-258; Denekamp, 1991, Int. J. Radiat. Biol. 60:401-408; Reed et al., 1989, Radiat. Res. 119:542-552).

A temporal increase in vascular leakage and permeability during PDT has been suggested in the transient pre-thrombosis or vascular stasis phase under conditions designed to cause irreversible tissue damage. (Sigdestad et al., 1996, Br. J. Cancer Suppl. 27:S89-92; Fingar, 1996, J. Clin. Laser Med. Surg. 14:323-328; Henderson et al., 1992, Photochem. Photobiol. 55:145-157; Reed et al., 1989, Radiat. Res. 119:542-552; Reed et al., 1989, J. Urol. 142:865-868; Wu et al., 1999, Curr. Opin. Ophthalmol. 10:217-220; de Vree et al., 1996, Cancer Res. 56:2908-2911; Fingar, 1996, J. Clin. Laser Med. Surg. 14:328-328; Sigdestad et al., 1996, Br. J. Cancer Suppl. 27:S89-92; Bellnier et al., 1995, Photochem. Photobiol. 62:896-905; Fingar et al, 1993, Photochem. Photobiol. 58:393-399; Fingar et al, 1993, Photochem. Photobiol. 58:251-258; Taber et al., 1993, Photochem. Photobiol. 57:856-861; ten Tije et al., 1999, Photochem. Photobiol. 69:494-499; Kerdel et al., 1987, J. Invest. Dermatol. 88:277-280; Fingar et al., 1997, Photochem. Photobiol. 66:513-517). However, due to the severe damage caused to the host organism, this phase during early tissue breakdown cannot be used for drug delivery for therapy or diagnosis. A temporal (i.e., pre-tissue/vessel-ablation) increase in vascular leakage and permeability has also been suggested during laser-induced hyperthermia alone and in combination with PDT (Liu et al., 1997, Cancer Lett. 111:157-165).

However, no methods have been designed that use radiation for targeted increase of vascular permeability for the delivery of therapeutic and diagnostic drugs. The present invention provides such methods.

### SUMMARY OF THE INVENTION

The present invention relates to methods for the delivery of a drug to a selected site in an organism. Using the described methods, one can deliver a drug to a tissue or organ of interest in any organism, for example, a human. Thus, the described methods facilitate the delivery of a therapeutic or diagnostic drug while using lower amounts of the drug. Furthermore, the methods facilitate the delivery of the drug to a site in an organism to which the drug may otherwise be difficult or impossible to deliver.

In accordance with certain embodiments of the invention, the methods of the invention induce increased vascular permeability in a selected site in an organism by supplying a photosensitizer to the organism and by irradiating the organism at the selected site. By supplying a drug to the organism when the radiation has induced increased vascular permeability at a specific site, the methods facilitate the delivery of the drug to the selected tissue or organ in the organism. In certain embodiments, the drug may be delivered from the bloodstream to the tissues and organs surrounding the blood vessel. In certain other embodiments, the drug may be delivered from a tissue or organ to a blood vessel and into the bloodstream.

In accordance with certain embodiments of the invention, the photosensitizer and the radiation can be used in the described methods so that a desired relative biological effect (RBE) is realized. In certain preferred embodiments, a RBE useful for the described method is sufficient to induce increased vascular permeability, yet insufficient to cause severe side effects, for example, thrombosis or vascular stasis.

In accordance with the invention, any drug can be delivered using the described methods. Drugs that can be delivered with the described methods may be of any size and any chemical nature or make-up, for example, nucleic acids, proteins, peptides, organic molecules, lipids, glycolipids, sugars, glycoproteins, etc.

### DETAILED DESCRIPTION

Methods of the Invention

The present invention relates to methods to deliver a drug to a selected site of an organism. As used herein, the terms “deliver” or “delivery,” when used in combination with a therapeutic or diagnostic drug, can refer to supplying a drug into a blood vessel of an organism so that the drug moves to a tissue and/or an organ surrounding the blood vessel. The terms “deliver” or “delivery” as used herein can also refer to supplying a drug to a tissue or an organ of an organism so that the drug moves to a blood vessel in or close to the tissue or organ. When a drug is delivered to a selected site using the described methods, the drug permeates into or out of a blood vessel at the site in an amount that is greater than the amount in which the drug would permeate into or out of a blood vessel at the site if a method of the invention was not employed. The increase in the amount of the drug that permeates into or out of a blood vessel at the selected site, in certain embodiments, is at least about 10 percent greater than the amount that the drug would permeate without using the method of the invention, more preferably at least about 20 percent, and even more preferably at least about 40 percent. In an especially preferred embodiment, the increase in drug permeability is at least about 100 percent, more preferably at least about 500 percent, even more preferably at least about 1,000 percent, more preferably at least about 5,000 percent, and most preferably at least about 10,000 percent. If the drug would not permeate a blood vessel without using the method of the present invention, then the amount of the drug that permeates the vessel when using the present invention, is at least 1 molecule, more preferably at least about 10 molecules, more preferably at least about 10² molecules, more preferably at least about 10³ molecules, more preferably at least about 10⁵ molecules, more preferably at least about 10⁷ molecules, more preferably at least about 10¹⁰ molecules, more preferably at least about 10²⁰ molecules.

As used herein, the term “drug,” refers to a compound, composition, or other material that is intended to exert a therapeutic or diagnostic effect on the organism that is separate and distinct from the effect of facilitating delivery of the drug to a specific site in the organism. In certain preferred embodiments, a drug is not aspirin, a thromboxane inhibitor, hyperthermia, alpha-interferon, glucose, nitrogen mustard (e.g., topical nitrogen mustard), folic acid, tazarotene, chemotherapeutic agents, cis-platinum, adriamycin, methotrexate, MX2, 1-(4-amino-2-methyl-5-pyrimidinyl)-methyl-3-(2-chloroethyl)-3-nitrosurea hydrochloride (ACNU), melphalan, UFT, buthionine sulfoximine, radiotherapy, etoposide, bioreductive drugs, misonidazole, pimonidazole, metronidazole, nimorazole, RB6145, RSU1069, SR4233, mitomycin-C, RB90740, electroporation, iontophoresis, haematoporphyrin derivative, verapamil, N-(2-hydroxypropyl)methacrylamide copolymer-bound adriamycin, mycobacterium cell-wall extract, vitamin D3-binding protein-derived macrophage-activating factor, the indoloquinone EO9, aluminum disulfonated phthalocyanine, electric current, ionizing radiation, thiotepa, Bacillus Calmette-Guerin (BCG), doxorubicin, x-rays.

As used herein, the term “selected site,” when used in connection with a tissue to which a drug is delivered with a method of the invention, means a portion of an organism to which the drug is delivered with the described methods. The portion of the organism, in certain embodiments, can be the entire organism.

As used herein, the term “organism” means an animal of any subspecies, species, genus, family, order, class, division, or lingdom. In a preferred embodiment, the organism is a human. In certain other embodiments, the organism is a mammal, a primate, a farm animal, a rodent, a bird, cattle, a cow, a mouse, a cat, a dog, a chimpanzee, a hamster, a fish, an ungulate, etc.

In the methods of the present invention, in certain embodiments, a photosensitizer is delivered to an organism followed by radiation of a selected site of the organism, so that vascular permeability at the selected site is increased. As used herein, the term “photosensitizer” means a molecule capable of increasing vascular permeability when used in the methods of the invention. In certain preferred embodiments, the radiation is applied soon after the photosensitizer has been introduced into the organism, for example, within 96 hours, more preferably within 48 hours, more preferably within 24 hours, more preferably within 12 hours, more preferably within 6 hours, more preferably within 3 hours, more preferably within 2 hours, more preferably within 1 hour, more preferably within 30 minutes, more preferably within 15 minutes, more preferably within 5 minutes, and most preferably immediately.

In certain preferred embodiments of the described methods, a transient increase in vascular permeability facilitates the transfer of a drug from the intravascular space to the extravascular tissue spaces and across membranes into cells of surrounding tissues and organs. This results in localized offloading of a drug or drugs in targeted zones of radiation.

In certain preferred embodiments, the methods of the invention are used to deliver a drug without exerting a substantial undesired side effect in the organism, more preferably without exerting a measurable undesired side effect. In certain embodiments, such an undesired side effect is, for example, thrombosis, vascular stasis, vascular breakdown, establishment of thrombogenic sites within blood vessel lumen, platelet aggregation, release of vasoactive molecules, leukocyte adhesion, vessel constriction, blood flow stasis, release of vasoactive eicosanoids during photodynamic therapy, vasoconstriction or vasodilation, endothelial cell damage, smooth muscle cell damage, stimulation of an acute immune response, altered expression of one or more genes involved in hemostasis, blood clotting, platelet aggregation/manufacture (see, e.g., Fingar, 1996, J. Clinical Laser Medicine & Surgery 14:323-328; Brasseur et al., 1996, Photochem. Photobiol. 64:702-706; McMahon et al., 1994, Cancer Res. 54:5374-5379; Tsilimbaris et al., 1994, Lasers. Surg. Med. 15:19-31; Fingar et al., 1993, Photochem. Photobiol. 58:393-399; Fingar et al., 1993, Photochem. Photobiol. 58:251-258; Reed et al., 1989, Radiat. Res. 119:542-552).

In certain preferred embodiments of the disclosed methods, a photosensitizer is supplied into the bloodstream of an organism. Following the supply of the photosensitizer into the bloodstream, a selected site of the organism is subjected to radiation. The drug of interest preferably is supplied to the irradiated site prior to or during the period of increased vascular permeability.

In certain other embodiments of the methods of the invention, a photosensitizer is supplied to a limited area in the organism, followed by radiation, and then supply of the drug. For example, the photosensitizer may be supplied in a localized manner into a tissue, for example, into a muscle, into adipose tissue, into connective tissue, into cartilage tissue, into nervous tissue, into skin, etc.

In accordance with the invention, a drug can be supplied to the organism for site specific delivery using the disclosed methods at any time so that it can be delivered to the desired site. For example, the drug can be supplied to the organism before radiation. Or, for example, the drug can be delivered shortly after radiation.

In certain embodiments, the drug is supplied into the bloodstream of an organism for site specific delivery. Following radiation in the disclosed methods, for example, the drug is delivered to the tissue surrounding irradiated blood vessels.

In certain other embodiments, the drug is supplied to a tissue of an organism for site specific delivery, for example, into a muscle, into adipose tissue, into connective tissue, into cartilage tissue, into nervous tissue, into skin, etc.

Photosensitizers Useful for the Described Methods

A variety of molecules can be used as a photosensitizer in the methods of the invention. In certain preferred embodiments, a photosensitizer useful for the methods of the invention is a molecule capable of increasing vascular permeability when it is supplied to an organism and irradiated. In certain other embodiments, more than one photosensitizer can be used in the described methods.

In certain other embodiments, a photosensitizer useful for the methods of the invention is capable of absorbing electromagnetic radiation and transferring that energy by a chemical process to desired target molecules, to biological complexes and/or cellular or tissue structures. Such an energy transfer may occur in a manner similar to photosynthesis in plants.

In certain embodiments, photosensitizers useful for the described methods include, but are not limited to, pyrrole derived macrocyclic compounds, naturally occurring or synthetic porphyrins and derivatives thereof naturally occurring or synthetic chlorins and derivatives thereof, naturally occurring or synthetic bacteriochlorins and derivatives thereof, naturally occurring or synthetic isobacteriochlorins and derivatives thereof, naturally occurring or synthetic phthalocyanines and derivatives thereof, naturally occurring or synthetic naphthalocyanines and derivatives thereof, naturally occurring or synthetic porphycenes and derivatives thereof, naturally occurring or synthetic porphycyanines and derivatives thereof, naturally occurring or synthetic pentaphyrins and derivatives thereof, naturally occurring or synthetic sapphyrins and derivatives thereof, naturally occurring or synthetic benzochlorins and derivatives thereof, naturally occurring or synthetic chlorophylls and derivatives thereof, naturally occurring or synthetic azaporphyrins and derivatives thereof, the metabolic porphyrinic precusor 5-amino levulinic acid and any naturally occurring or synthetic derivatives thereof, photofrin™, synthetic diporphyrins and dichlorins, O-substituted tetraphenyl porphyrins (picket fence porphyrins), 3,1-meso tetrakis (o-propionamido phenyl) porphyrin, verdins, purpurins (e.g., tin and zinc derivatives of octaethylpurpurin (NT2), and etiopurpurin (ET2)), zinc naphthalocyanines, anthracenediones, anthrapyrazoles, aminoanthraquinone, phenoxazine dyes, chlorins (e.g., chlorin e6, and mono-1-aspartyl derivative of chlorin e6), benzoporphyrin derivatives (BPD) (e.g., benzoporphyrin monoacid derivatives, tetracyanoethylene adducts of benzoporphyrin, dimethyl acetylenedicarboxylate adducts of benzoporphyrin, Diels-Adler adducts, and monoacid ring “a” derivative of benzoporphyrin), low density lipoprotein mediated localization parameters similar to those observed with hematoporphyrin derivative (HPD), sulfonated aluminum phthalocyanine (Pc) (sulfonated AIPc, disulfonated (AlPcS.sub.2), tetrasulfonated derivative, sulfonated aluminum naphthalocyanines, chloroaluminum sulfonated phthalocyanine (CASP)), phenothiazine derivatives, chalcogenapyrylium dyes cationic selena and tellurapyrylium derivatives, ring-substituted cationic PC, pheophorbide alpha, hydroporphyrins (e.g., chlorins and bacteriochlorins of the tetra(hydroxyphenyl) porphyrin series), phthalocyanines, hematoporphyrin (BP), protoporphyrin, uroporphyrin III, coproporphyrin III, protoporphyrin IX, 5-amino levulinic acid, pyrromethane boron difluorides, indocyanine green, zinc phthalocyanine, dihematoporphyrin (514 nm), benzoporphyrin derivatives, carotenoporphyrins, hematoporphyrin and porphyrin derivatives, rose bengal (550 nm), bacteriochlorin A (760 nm), epigallocatechin, epicatechin derivatives, hypocrellin B, urocanic acid, indoleacrylic acid, rhodium complexes, etiobenzochlorins, octaethylbenzochlorins, sulfonated Pc-naphthalocyanine, silicon naphthalocyanines, chloroaluminum sulfonated phthalocyanine (610 nm), phthalocyanine derivatives, iminium salt benzochlorins and other iminium salt complexes, Merocyanin 540, Hoechst 33258, and other DNA-binding fluorochromes, psoralens, acridine compounds, suprofen, tiaprofenic acid, non-steroidal anti-inflammatory drugs, methylpheophorbide-a-(hexyl-ether) and other pheophorbides, furocoumarin hydroperoxides, Victoria blue BO, methylene blue, toluidine blue, porphycene compounds as described in U.S. Pat. No. 5,179,120 (the entire contents of which are herein incorporated by reference), indocyanines, and any other photosensitizers, and any combination of any or all of the above. A few of the light frequencies to which the photosensitizers are sensitive are provided in parenthesis.

As used herein, the terms “derivative” or “derivatives” mean molecules with chemical groups having functionality that are attached covalently or non-covalently to the molecule. Examples of the functionality are: (1) hydrogen; (2) halogen, such as fluoro, chloro, iodo and bromo; (3) lower allyl, such as methyl, ethyl, n-propyl, isopropyl, t-butyl, n-pentyl and the like groups; (4) lower alkoxy, such as methoxy, ethoxy, isopropoxy, n-butoxy, tentoxy and the like; (5) hydroxy; alkylhydroxy, alkylethers (6) carboxylic acid or acid salts, such as —CH₂COOH, —CH₂COO⁻Na⁺, —CH₂CH₂COOH, —CH₂CH₂COONa, —CH₂CH₂CH(Br)COOH, —CH₂CH₂CH(CH₃)COOH, —CH₂CH(Br)COOH, —CH₂CH(CH₃)COOH, —CH(CI)—CH₂—CH(CH₃)—COOH, —CH₂—CH₂—C(CH₃)₂—COOH, —CH₂—CH₂—C(CH₃)₂—COO⁻K⁺, —CH₂—CH₂—CH₂—CH₂—COOH, C(CH₃)₃—COOH, CH(CI)₂COOH and the like; (7) carboxylic acid esters, such as —CH₂CH₂COOCH₃, —CH₂CH₂COOCH₂CH₃, —CH₂CH(CH₃)COOCH₂CH₃, —CH₂CH₂CH₂COOCH₂CH₂CH₂C H₃, —CH₂CH(CH₃)₂COOCH₂CH₃, and the like; (8) sulfonic acid or acid salts, for example, group I and group 11 salts, ammonium salts, and organic cation salts such as alkyl and quaternary ammonium salts; (9) sulfonylamides such as substituted and unsubstituted benzene sulfonamides; (10) sulfonic acid esters, such as methyl sulfonate, ethyl sulfonate, cyclohexyl sulfonate and the like; (11) amino, such as unsubstituted primary amino, methylamino, ethylamino, n-propylamino, isopropylamino, 5-butylamino, secbutylamino, dimethylamino, trimethylamino, diethylamino, triethylamino, di-n-propylamino, methylethylamino, dimethyl-sec-butylamino, 2-aminoethanoxy, ethylenediamino, 2-(N-methylamino) heptyl, cyclohexylamino, benzylamino, phenylethylamino, anilino, -methylanilino, N,N-dimethylanilino, N-methyl-N ethylanilino, 3,5-dibromo-4-anilino, p-toluidino, diphenylamino, 4,4′-dinitrodiphenylamino and the like; (12) cyano; (13) nitro; (14) a biologically active group; (15) any other substituent that increases the amphiphilic nature of the compounds; or (16) doso- or nido-carborane cages.

The term “biologically active group” can be any group that selectively promotes the accumulation, elimination, binding rate, or tightness of binding in a particular biological environment. For example, one category of biologically active groups is the substituents derived from sugars, specifically, (1) aldoses such as glyceraldehyde, erythrose, threose, ribose, arabinose, xylose, lyxose, allose, altrose, glucose, mannose, gulose, idose, galactose, and talose; (2) ketoses such as hydroxyacetone, erythrulose, rebulose, xylulose, psicose, fructose, verbose, and tagatose; (3) pyranoses such as glucopyranose; (4) furanoses such as fructo-furanose; (5) O-acyl derivatives such as penta-O-acetyl-a-glucose; (6) O-methyl derivatives such as methyl a-glucoside, methyl p-glucoside, methyl a-glucopyranoside and methyl-2,3,4,6-tetra-O-methyl glucopyranoside; (7) phenylosazones such as glucose phenylosazone; (8) sugar alcohols such as sorbitol, mannitol, glycerol, and myo-inositol; (9) sugar acids such as gluconic acid, glucaric acid and glucuronic acid, o-gluconolactone, 5-glucuronolactone, ascorbic acid, and dehydroascorbic acid; (10) phosphoric acid esters such as a-glucose 1-phosphoric acid, a-glucose 6-phosphoric acid, a-fructose 1,6-diphosphoric acid, and a-fructose 6-phosphoric acid; (11) deoxy sugars such as 2-deoxy-ribose, rhammose (deoxy-mannose), and fructose (6-deoxy-galactose); (12) amino sugars such as glucosamine and galactosamine; muramic acid and neurarninic acid; (13) disaccharides such as maltose, sucrose and trehalose; (14) trisaccharides such as raffinose (fructose, glucose, galactose) and melezitose (glucose, fructose, glucose);(15) polysaccharides (glycans) such as glucans and mannans; and (16) storage polysaccharides such as a-amylose, amylopectin, dextrins, and dextrans.

Amino acid derivatives are also useful biologically active substituents, such as those derived from valine, leucine, isoleucine, threonine, methionine, phenylalanine, tryptophan, alanine, arginine, aspartic acid, cysteine, cysteine, glutamic acid, glycine, histidine, proline, serine, tyrosine, asparagine and glutamine. Also useful are peptides, particularly those known to have affinity for specific receptors, for example, oxytocin, vasopressin, bradykinin, LHRH, thrombin and the like.

Another useful group of biologically active substituents are those derived from nucleosides, for example, ribonucleosides such as adenosine, guanosine, cytidine, and uridine; and 2′-deoxyribonucleosides, such as 2′-deoxyadenosine, 2′-deoxyquanosine, 2′-deoxycytidine, and 2′-deoxythymidine.

Another category of biologically active groups that is particularly useful is any ligand that is specific for a particular biological receptor. The term “ligand specific for a receptor” refers to a moiety that binds a receptor at cell surfaces, and thus contains contours and charge patterns that are complementary to those of the biological receptor. The ligand is not the receptor itself, but a substance complementary to it. It is well understood that a wide variety of cell types have specific receptors designed to bind hormones, growth factors, or neurotransmitters. However, while these embodiments of ligands specific for receptors are known and understood, the phrase “ligand specific for a receptor”, as used herein, refers to any substance, natural or synthetic, that binds specifically to a receptor.

Examples of such ligends include: (1) the steroid hormones, such as progesterone, estrogens, androgens, and the adrenal cortical hormones; (2) growth factors, such as epidermal growth factor, nerve growth factor, fibroblast growth factor, and the like; (3) other protein hormones, such as human growth hormone, parathyroid hormone, and the like; (4) neurotransmitters, such as acetylcholine, serotonin, dopamine, and the like; and (5) antibodies. Any analog of these substances that also succeeds in binding to a biological receptor is also included. Particularly useful examples of substituents tending to increase the amphiphilic nature of the photosensitizer include: (1) long chain alcohols, for example, —C₁₂H₂₄—OH where —C₁₂H₂₄ is hydrophobic; (2) fatty acids and their salts, such as the sodium salt of the long-chain fatty acid oleic acid; (3) phosphoglycerides, such as phosphatidic acid, phosphatidyl ethanolamine, phosphatidyl choline, phosphatidyl serine, phosphatidyl inositol, phosphatidyl glycerol, phosphatidyl 3′-O-alanyl glycerol, cardiolipin, or phosphatidal choline; (4) sphingolipids, such as sphingomyelin; and (5) glycolipids, such as glycosyidiacylglycerols, cerebrosides, sulfate esters of cerebrosides or gangliosides.

In certain embodiments, photosensitizers useful for the described methods include, but are not limited to, members of the following classes of compounds: porphyrins, chlorins, bacteriochlorins, purpurins, phthalocyanines, naphthalocyanines, texaphyrines, and non-tetrapyrrole photosensitizers. For example, the photosensitizer may be, but is not limited to, Photofrin®, benzoporphyrin derivatives, tin ethyl etiopurpurin (SnET2), sulfonated chloroaluminum phthalocyanines and methylene blue, and any combination of any or all of the above.

In certain other embodiments, any compound, molecule, ion, or atom can be examined for its usefulness for the described methods, for example, by testing it in the hamster model described in the Examples Section below. Other animal models known in the art can also be used to test a photosensitizer for its usefulness in the described methods. Such animal models are described in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

See, also, U.S. Pat. Nos. 5,965,598; 5,952,329; 5,942,534; 5,913,884; 5,866,316; 5,775,339; 5,773,460; 5,637,451; 5,556,992; 5,514,669; 5,506,255; 5,484,778; 5,459,159; 5,446,157; 5,409,900; 5,407,808; 5,389,378; 5,368,841; 5,330,741; 5,314,905; 5,298,502; 5,298,018; 5,286,708; 5,262,401; 5,244,671; 5,238,940; 5,214,036; 5,198,460; 5,190,966; 5,179,120; 5,173,504; 5,171,741; 5,166,197; 5,132,101; 5,064,952; 5,053,423; 5,047,419; 4,968,715, which describe photosensitizers useful in the described methods.

Dosage of Photosensitizers

A photosensitizer is used in the disclosed methods at a dosage that facilitates the increase of vascular permeability to deliver a drug of interest. A useful dosage of a photosensitizer for the disclosed methods depends, for example, on a variety of properties of the activating light (e.g., wavelength, energy, energy density, intensity), the optical properties of the target tissue and properties of the photosensitizer.

Within the field of radiobiology, and useful to determine dosages of photosensitizers and radiation for the methods of the invention, the concept of relative biological effectiveness (RBE) is used to measure the relative efficacy in differing tissues of various kinds or wavetypes of the activating radiation. The RBE value obtained in a method of the invention gives the stringency of the conditions employed. The concept of RBE is known to those skilled in the art, and is discussed in, Kraft, 1999, Strahlenther Onkol. 175 S2:44-47; Hawkins, 1998, Med. Phys. 25:1157-1170; Tanaka et al., 1994, Mutat. Res. 323:53-61; MacVittie et al., 1991, Radiat. Res. 128:S29-36; Star et al., 1990, Photochem. Photobiol. 52:547-554; Morgan et al., 1988, Br. J. Radiol. 732:1127-1135; Star, 1997, Phys. Med. Biol. 42:763-787; Marijnissen et al., 1996, Phys. Med. Biol. 41:1191-1208; Marijnissen et al., 1993, Phys. Med. Biol. 38:567-582. RBE describes the biological potency of the treatment, in this case using a photosensitizer and radiation combination. Quantitation of the RBE allows determination of equivalent potencies to be calculated for treatments using other photosensitizer and radiation combinations, as well as allowing equivalent doses of the treatment to be determined for other tissues and other organisms.

The RBE can be expressed, for example, as the amount of radiation of a certain energy which will produce a specified biological effect in a target tissue relative to the amount of radiation of a different energy which will produce the same biological effect in the same target tissue. The RBE between two energies of radiation may thus vary depending on the target tissue or organ. According to what is known in the field of photodynamic therapy, and useful for the present invention, the biological effect is the product of the amount of radiation and the amount of photosensitizer present in the target tissue at the time of the activation by light. This is referred to as “reciprocity”. To equate this product to the radiobiological concept of RBE, modifying factors are used to describe the ability of the photosensitizer to absorb the activating light (i.e., its absorbance or extinction co-efficient at the wavelength of the activating light), the ability of the photosensitizer to photo-chemically convert the activating light into chemical energy which mediates the biological effect (the triplet “manifold”, or the “potency” of the photosensitizer) and the ability of the light to pass through the tissue to activate the photosensitizer. When employing the RBE concept, it is preferred that the photosensitizer is homogeneously distributed within the target field or tissue, and that the light distribution within the target field or tissue is isotropic.

The RBE is defined by the equation RBE=a·f·c, wherein “a” equals the concentration of the photosensitizer at a given time, “f” equals the amount of electromagnetic radiation which interacts with the photosensitizer (this term is a product of the absorption coefficient of the photosensitizer at the wavelength of activation and the total light dose delivered), and “c” equals a propotionality constant which may vary between different cells, tissues or target zones.

Thus, the same biological effect can be achieved using either low photosensitizer doses activated by high light doses, or high photosensitizer doses activated by low light doses. This principle is referred to as “reciprocity.” Reciprocity may not hold at the extremes of very high drug doses in combination with very low light doses, or very low photosensitizer doses in combination with very high light doses. Furthermore, the end biological effect can vary with different wavelengths of activating electromagnetic radiation. For example, a photosensitizer may not have a high absorption coefficient at a given wavelength, and thus the light dose required to achieve the desired effect will need to be greater than when using a wavelength where the photosensitizer has a high absorption coefficient.

An example of how this is used is provided in the following references describing the photodynamic destruction mediated by two photosensitizers, a boronated protoporphyrin (BOPP) Hill et al., 1992, Proc. Natl. Acad. Sci. 89:1785-1789; Hill et al., 1995, Proc. Natl. Acad. Sci. 92:12126-12130) and Hematoporphyrin derivative (HpD) (Kaye et al., 1985, Neurosurgery 17:883-890; Kaye et al., 1987, Neurosurgery 20:408-415) in a brain tumor model in rats and mice. The tissue distribution and plasma pharmacokinetics were determined in the same animal models for both photosensitizers, as was the ability of both photosensitizers to mediate photodynamic tumor destruction. in the same animal models. Thus, comparative assessments could be determined. In these examples, the calculation of the RBE was simplified because both photosensitizers were activated with the same wavelength of light (630 nm), and the same tissue/tumor model was used. In the cited examples the RBE of BOPP relative to HpD was determined to be between 0.05-0.1. Thus BOPP was determined to be a more potent photosensitizer than HpD.

Assays used in the above example can be used to determine the RBE for varying drugs, in varying target tissue of interest. Those skilled in the art have made use of a wide range of cell culture, animal and human models to determine the most optimal dosimetry of light and photosensitizer for a given target (for reviews see, e.g., McCaughan, 1999, Drugs Aging 15:49-68; Dougherty et al., 1998, J. Natl. Cancer Inst. 90:889-905).

In certain embodiments, the RBE value employed is sufficient to result in increased vascular permeability at the selected site in the organism of interest. In certain preferred embodiments, the RBE value employed is sufficient to result in increased vascular permeability at the selected site in the organism of interest to deliver the drug of interest. In yet certain other embodiments, the RBE value employed is sufficient to result in increased vascular permeability at the selected site in the organism of interest to deliver the drug of interest at a rate and/or in an amount sufficient to accomplish the therapeutic or diagnostic objective of interest, for example, sufficient to treat a disease condition of interest.

The RBE value useful for the delivery of a drug of interest can be determined, for example, by using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; Ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

In certain embodiments, the blood level dose of the photosensitizer used in the disclosed methods is from about 0.1 nanomole of photosensitizer per ml of blood (nmole/ml) to about 100 micromole of photosensitizer per ml of blood (μmole/ml), more preferably from about 0.15 nmole/ml to about 80 μmole/ml, more preferably from about 0.2 nmole/ml to about 60 μmole/ml, more preferably from about 0.3 nmole/ml to about 40 μmole/ml, more preferably from about 0.5 nmole/ml to about 20 μmole/ml, more preferably from about 1 nmole/ml to about 1 μmole/ml, more preferably from about 2 nmole/ml to about 500 nmole/ml, more preferably from about 5 nmole/ml to about 250 nmole/ml, more preferably from about 10 nmole/ml to about 100 nmole/ml, more preferably from about 20 nmole/ml to about 50 nmole/ml, and most preferably from about 30 nmole/ml to about 40 nmole/ml.

In certain embodiments, the blood level dose of the photosensitizer used in the disclosed methods is from about 0.1 nanomole of photosensitizer per ml of blood (nmole/ml) to about 1 micromole of photosensitizer per ml of blood (μmole/ml), more preferably from about 0.125 nmole/ml to about 600 nmole/ml, more preferably from about 0.15 nmole/ml to about 300 nmole/ml, more preferably from about 0.25 nmole/ml to about 150 nmole/ml, more preferably from about 0.4 nmole/ml to about 75 nmole/ml, more preferably from about 0.8 nmole/ml to about 35 nmole/ml, more preferably from about 1.5 nmole/ml to about 25 nmole/ml, more preferably from about 2.5 nmole/ml to about 15 nmole/ml, more preferably from about 3.5 nmole/ml to about 10 nmole/ml, more preferably from about 4 nmole/ml to about 6 nmole/ml, and most preferably about 5 nmole/ml.

In certain embodiments, the tissue level dose of the photosensitizer used in the disclosed methods is from about 0.1 nanomole of photosensitizer per g of tissue wet weight (nmole/g) to about 100 nanomole of photosensitizer per g of tissue wet weight (nmole/g), more preferably from about 0.125 nmole/g to about 80 nmole/g, more preferably from about 0.15 nmole/g to about 60 nmole/g, more preferably from about 0.25 nmole/g to about 40 nmole/g, more preferably from about 0.4 nmole/g to about 20 nmole/g, more preferably from about 0.8 nmole/g to about 15 nmole/g, more preferably from about 1.5 nmole/g to about 10 nmole/g, more preferably from about 2.5 nmole/g to about 5 nmole/g, and most preferably from about 3.5 nmole/g.

In certain other embodiments, the dose of the photosensitizer used in the disclosed methods is from about 0.5 microgram of photosensitizer per kilogram of body weight (i.e., the body weight of the organism or patient) (μg/kg) to about 10 milligram of photosensitizer per kilogram of body weight (mg/kg), more preferably from about 1 μg/kg to about 6 mg/kg, more preferably from about 2 μg/kg to about 3 mg/kg, more preferably from about 4 μg/kg to about 1.5 mg/kg, more preferably from about 8 μg/kg to about 0.75 mg/kg, more preferably from about 20 μg/kg to about 350 μg/kg, more preferably from about 40 μg/kg to about 200 μg/kg, more preferably from about 60 μg/kg to about 100 μg/kg, and most preferably about 80 μg/kg.

The concentration of a photosensitizer in an animal, patient, or any kind of sample, may be determined by any means known in the art including, but not limited to, fluorescent spectroscopy, HPLC, PET, quantitative MRI, radio-labeling, immunohistochemistry, IR spectroscopy, Raman spectroscopy, Tyndall scattering.

The dosage of a photosensitizer useful for the described methods can be determined, for example, by using the animal model described in detail in the Examples Section below. Other animal models are known to the skilled artisan and are discussed in, for example, Bellnier et al, 1995, Photochemistry and Photobiology 62:896-905; Endrich et al, 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999,J. Med. Chem. 42:4405-4413; Baccichetti, et al., 1992, Farmaco. 47:1529-1541; Roberts et al., 1989, Photochem. Photobiol. 49:431-438.

Photosensitizer Toxicity

In accordance with the preferred embodiments of the present invention, a photosensitizer is used in the described methods at a dosage less than the dosage that would be so toxic on the organism of interest as to render the described methods unfeasible. Specifically, toxic effects exerted by the photosensitizer at the selected dosage preferably are nonlethal to the organism.

In accordance with the preferred embodiments of the invention, the photosensitizer is used at a dosage so that in combination with the selected radiation dose no toxic effects are exerted on the organism that render the described methods unfeasible. Specifically, toxic effects exerted by the photosensitizer at the selected dosage of radiation preferably are nonlethal to the organism.

In certain preferred embodiments, the described methods are used with photosensitizer dosages so as to minimize undesirable effects, for example, thrombosis, vascular stasis, vascular breakdown, establishment of thrombogenic sites within blood vessel lumen, platelet aggregation, release of vasoactive molecules, leukocyte adhesion, vessel constriction, blood flow stasis, mitochondrial injury, lysozome injury, mutagenicity, carcinogenicity, fibrosis, inflammation, neurotoxicity, hyperpigmentation, smooth muscle cell hypertrophy, immunotoxicity, sensitivity with other light-reactive agents (antibiotics such as fluoroquinones, tetracycline-derivatives; chemotherapeutics such as adriamycin, 5-FU) (see, e.g., Fingar, 1996, J. Clinical Laser Medicine & Surgery 14:323-328; Brasseur et al., 1996, Photochem. Photobiol. 64:702-706; McMahon et al., 1994, Cancer Res. 54:5374-5379; Tsilimbaris et al., 1994, Lasers. Surg. Med. 15:19-31; Fingar et al., 1993, Photochem. Photobiol. 58:393-399; Fingar et al., 1993, Photochem. Photobiol. 58:251-258; Reed et al., 1989, Radiat. Res. 119:542-552).

Toxicological data for many photosensitizers are known in the art. See, for example, Ouedraogo et al., 1999, Photochem. Photobiol. 70:123-129; Hadkiotis et al., 1999, Mutagenesis 14:193-198; Murrer et al., 1999, Br. J. Cancer 80:744-755; Mandys et al., 1998, Photochem. Photobiol. 47:197-201; Muller et al., 1998, Toxicol. Lett. 102-103:383-387; Waterfield et al., 1997, Immunopharmacol. Immunotoxicol 19:89-103; Munday et al, 1996, Biochim. Biophys. Acta 1311:1-4; Noske et al., 1995, Photochem. Photobiol. 61:494-498; Lovell et al., 1992, Food Chem. Toxicol 30:155-160.

The toxicity of a photosensitizer at any dosage can be determined using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

Supply of Photosensitizer

A photosensitizer useful for the described methods may be supplied to the organism of interest by any means known to the skilled artisan including, but not limited to, oral, local, slow release implant, systemic injection (e.g., venous, arterial, lymphatic), local injection (e.g., slow release formulations), hydrogel polymers, inhalation delivery (e.g., dry powder, particulates), electroporation-mediated, iontophoresis or electrophoresis- mediated, microspheres or nanospheres, liposomes, erythrocyte shells, implantable delivery devices, local drug delivery catheter, perivascular delivery, pericardial delivery, eluting stent delivery.

A photosensitizer useful for the described methods may be prepared or formulated for supply to the organism of interest in any medium known to the skilled artisan including, but not limited to, tablet, solution, gel, aerosol, dry powder, biomolecular matrix, inhalation.

See, also, U.S. Pat. Nos. 5,965,598; 5,952,329; 5,942,534; 5,913,884; 5,866,316; 5,775,339; 5,773,460; 5,637,451; 5,556,992; 5,514,669; 5,506,255; 5,484,778; 5,459,159; 5,446,157; 5,409,900; 5,407,808; 5,389,378; 5,368,841; 5,330,741; 5,314,905; 5,298,502; 5,298,018; 5,286,708; 5,262,401; 5,244,671; 5,238,940; 5,214,036; 5,198,460; 5,190,966; 5,179,120; 5,173,504; 5,171,741; 5,166,197; 5,132,101; 5,064,952; 5,053,423; 5,047,419; 4,968,715, which describe the supply and formulation of photosensitizers useful in the described methods.

Radiation

In accordance with the invention, the organism, to which the photosensitizer is supplied in the described methods, is irradiated. In certain preferred embodiments, the radiation used in the described methods is electromagnetic radiation.

The radiation used in the described methods, in certain embodiments, is calibrated so that it enhances vascular permeability at the selected site in the organism of interest when applied to the chosen type and dose of photosensitizer. Radiation used in the described methods is preferably calibrated, for example, by choosing the appropriate wavelength, power, power density, energy density, and time of application relative to the time of supply of the photosensitizer to the organism.

In certain preferred embodiments, radiation used in the described methods is calibrated in such a way as to yield a desired RBE value. Preferably, the radiation used in the described methods is calibrated so that the desired RBE value is realized according to the principle of reciprocity.

See, also, U.S. Pat. Nos. 6,013,053; 6,011,563; 5,976,175; 5,971,918; 5,961,543; 5,944,748; 5,910,510; 5,849,027; 5,845,640; 5,835,648; 5,817,048; 5,798,523; 5,797,868; 5,793,781; 5,782,895; 5,707,401; 5,571,152; 5,533,508; 5,489,279; 5,441,531; 5,344,434; 5,219,346; 5,146,917; 5,054,867, which describe radiation techniques useful for the described methods.

Wavelength of Radiation

In accordance with the invention, the radiation used in the described methods has a wavelength that, in combination with the photosensitizer, facilitates the increase of vascular permeability at the selected site of the organism of interest. Preferably, the radiation wavelength facilitates increased vascular permeability for the drug of interest.

In certain preferred embodiments, the wavelength used in the described methods is chosen in view of the reciprocity principle to obtain a desirable RBE value. For example, if a photosensitizer has a low absorption coefficient at a given wavelength, the light dose typically required to achieve the desired effect is greater, possibly much greater, than when using a wavelength where the photosensitizer has a high absorption coefficient.

In certain other embodiments, the wavelength is chosen so that the toxicity to the organism is maintained at a level that does not prohibit the application of the described methods, preferably at a low level, and most preferably at a minimal level.

In certain embodiments, the radiation wavelength used in the described methods is absorbed by the photosensitizer used. In certain preferred embodiments, the radiation wavelength used in the described methods is such that the absorption coefficient at the chosen wavelength for the photosensitizer used is at least about 20 percent of the highest absorption coefficient for that photosensitizer on the spectrum of electromagnetic radiation of from about 280 nm to about 1700 nm, more preferably at least about 40 percent, more preferably at least about 60 percent, more preferably at least about 80 percent, more preferably at least about 90 percent, and most preferably about 100 percent. In certain other embodiments, the radiation wavelength used in the described methods is such that the absorption coefficient at the chosen wavelength for the photosensitizer used is from about 5 percent to about 100 percent of the highest absorption coefficient for that photosensitizer on the spectrum of electromagnetic radiation of from about 280 nm to about 1700 mu, more preferably from about 10 percent to about 95 percent. If more than one photosensitizer is used in the described methods, the above values should apply to at least one of the photosensitizers used.

In certain other embodiments, the wavelength used in the described methods is from about 200 nm to about 2,000 nm, more preferably from about 240 nm to about 1,850 nm, more preferably from about 280 nm to about 1,700 nm, more preferably from about 330 nm to about 1,500 nm, more preferably from about 380 nm to about 1,250 nm, more preferably from about 430 nm to about 1,000 nm, more preferably from about 480 nm to about 850 nm, more preferably from about 530 nm to about 750 nm, more preferably from about 580 nm to about 700 nm, more preferably from about 600 nm to about 680 nm, more preferably from about 620 nm to about 660 nm, more preferably from about 640 nm to about 650 nm.

In certain embodiments, the wavelengths provided above are the wavelengths of the radiation used as it is emitted form the source of radiation used.

The wavelength of radiation useful for the described methods can be determined using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichett, et al., 1992, Farmaco. 47:1529-1541; Roberts et al., 1989, Photochem. Photobiol. 49:431-438.

Power of Radiation

In accordance with the invention, the power of the radiation used in the described methods facilitates the increase of vascular permeability at the selected site of the organism of interest. Preferably, the power of the radiation used facilitates increased vascular permeability for the drug of interest.

In certain other embodiments, the power of the radiation is chosen so that the toxicity to the organism is maintained at a level that does not prohibit the application of the described methods, preferably at a low level, and most preferably at a minimal level.

In certain other embodiments, the power of radiation used in the described methods is from about 1 mWatt (mW) to about 5 Watt (W), more preferably from about 2 mW to about 4 W, more preferably from about 4 mW to about 3 W, more preferably from about 8 mW to about 2 W, more preferably from about 20 mW to about 1.5 W, more preferably from about 40 mW to about 1 W, more preferably from about 100 mW to about 800 mW, more preferably from about 150 mW to about 650 mW, more preferably from about 200 mW to about 500 mW, more preferably from about 250 mW to about 400 mW, more preferably from about 300 mW to about 350 mW.

The power of radiation useful for the described methods can be determined using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti et al., 1992, Farmaco. 47:1529-1541; Roberts et al., 1989, Photochem. Photobiol. 49:431-438.

Power Density of Radiation

In accordance with the invention, the power density of the radiation used in the described methods facilitates the increase of vascular permeability at the selected site of the organism of interest. Preferably, the power density of the radiation used facilitates increased vascular permeability for the drug of interest.

In certain other embodiments, the power density of the radiation is chosen so that the toxicity to the organism is maintained at a level that does not prohibit the application of the described methods, preferably at a low level, and most preferably at a minimal level.

In certain other embodiments, the power of radiation used in the described methods is from about 0.01 mWatt/cm² (mW/cm²) to about 1,000 mW/cm², more preferably from about 0.05 mW/cm² to about 500 mW/cm², more preferably from about 0.1 mW/cm² to about 250 mW/cm², more preferably from about 0.2 mW/cm² to about 150 mW/cm², more preferably from about 0.5 mW/cm² to about 100 mW/cm², more preferably from about 1 mW/cm² to about 75 mW/cm², more preferably from about 2 mW/cm² to about 60 mW/cm², more preferably from about 5 mW/cm² to about 50 mW/cm², more preferably from about 10 mW/cm² to about 40 mW/cm², more preferably from about 20 mW/cm² to about 30 mW/cm², and most preferably about 25 mW/cm².

In certain preferred embodiments, the power density values provided above are measured at the target site of the organism.

The power of radiation useful for the described methods can be determined using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vase. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

Intensity/Energy Density of Radiation

In accordance with the invention, the intensity or energy density (intensity) of the radiation used in the described methods facilitates the increase of vascular permeability at the selected site of the organism of interest. Preferably, the intensity of the radiation used facilitates increased vascular permeability for the drug of interest.

In certain preferred embodiments, the intensity used in the described methods is chosen in view of the reciprocity principle to obtain a desirable RBE value. For example, if a photosensitizer is used at a low dose, the radiation intensity typically required to achieve the desired effect is greater, possibly much greater, than when using the photosensitizer at a higher dosage.

In certain other embodiments, the intensity of the radiation is chosen so that the toxicity to the organism is maintained at a level that does not prohibit the application of the described methods, preferably at a low level, and most preferably at a minimal level.

In certain other embodiments, the intensity of radiation used in the described methods is from about 0.05 Joule/cm² (J/cm²) to about 1,000 J/cm², more preferably from about 0.1 J/cm² to about 500 J/cm², more preferably from about 0.2 J/cm² to about 250 J/cm², more preferably from about 0.4 J/cm² to about 150 J/cm², more preferably from about 1 J/cm² to about 100 J/cm², more preferably from about 2 J/cm² to about 75 J/cm², more preferably from about 4 J/cm² to about 60 J/cm², more preferably from about 7.5 J/cm² to about 50 J/cm², more preferably from about 10 J/cm² to about 40 J/cm², more preferably from about 15 J/cm² to about 35 J/cm², more preferably from about 20 J/cm² to about 30 J/cm², and most preferably about 25 mW/cm².

In certain preferred embodiments, the intensity values provided above are measured at the target site of the organism.

The power of radiation useful for the described methods can be determined using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

Timing of Radiation

In accordance with the invention, the timing of the radiation used in the described methods relative to the supply of the photosensitizer (i.e., timing of radiation) facilitates the increase of vascular permeability at the selected site of the organism of interest. Preferably, the timing of radiation used facilitates increased vascular permeability for the drug of interest.

In certain other embodiments, the timing of radiation is chosen so that the toxicity to the organism is maintained at a level that does not prohibit the application of the described methods, preferably at a low level, and most preferably at a minimal level.

In certain other embodiments, the timing of radiation used in the described methods is from about 0 hours to about 168 hours post administration of the photosensitizer, more preferably from about 0.1 hours to about 120 hours, more preferably from about 0.2 hours to about 96 hours, more preferably from about 0.3 hours to about 72 hours, more preferably from about 0.4 hours to about 48 hours, more preferably from about 0.5 hours to about 36 hours, more preferably from about 0.6 hours to about 24 hours, more preferably from about 0.7 hours to about 12 hours, more preferably from about 0.8 hours to about 10 hours, more preferably from about 0.9 hours to about 8 hours, more preferably from about 1 hours to about 6 hours, more preferably from about 1.1 hours to about 4 hours, more preferably from about 1.2 hours to about 3 hours, more preferably from about 1.3 hours to about 2.5 hours, more preferably from about 1.4 hours to about 2 hours, more preferably from about 1.5 hours to about 1.8 hours, and most preferably about 1.6 hours.

In certain embodiments, the timing values provided above are measured from the time photosensitizer administration begins. In certain other embodiments, the timing values provided above are measured from the time photosensitizer administration ends. In certain embodiments, the timing values provided above are measured from the time 50 percent of the photosensitizer has been administered.

The timing of radiation useful for the described methods can be determined using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

Radiation Toxicity

In accordance with the invention, radiation is used in the described methods at a dosage that does not exert such toxic effects on the organism of interest so that the described methods are rendered unfeasible. Specifically, toxic effects exerted by the radiation at the selected dosage preferably are nonlethal to the organism.

In certain embodiments, radiation is used in the described methods so that no undesirable thermal effects or skin effects are caused.

In certain other embodiments, the radiation is used at a dosage so that, in combination with the selected photosensitizer dose, no toxic effects are exerted that render the described methods unfeasible. Specifically, toxic effects exerted by the radiation at the selected dosage of the photosensitizer preferably are nonlethal to the organism.

In certain preferred embodiments, the described methods are used with radiation dosages so to minimize undesirable effects, for example, thrombosis, vascular stasis, vascular breakdown, establishment of thrombogenic sites within blood vessel lumen, platelet aggregation, release of vasoactive molecules, leukocyte adhesion, vessel constriction, blood flow stasis, edema, erythema, fibrosis, ischemia, photosensitivity, pain, vasoconstriction, spontaneous human combustion (see, e.g., Fingar, 1996, J. Clinical Laser Medicine & Surgery 14:323-328; Brasseur et al., 1996, Photochem. Photobiol. 64:702-706; McMahon et al., 1994, Cancer Res. 54:5374-5379; Tsilimbaris et al., 1994, Lasers. Surg. Med. 15:19-31; Fingar et al., 1993, Photochem. Photobiol. 58:393-399; Fingar et al., 1993, Photochem. Photobiol. 58:251-258; Reed et al., 1989, Radiat. Res. 119:542-552).

Toxicological data for radiation at various wavelengths and intensities are known in the art.

The toxicity of radiation at any dosage can be determined using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

Sources of Radiation

In accordance with the invention, any radiation source producing a wavelength that can activate the photosensitizer used can be employed in the described methods. In certain embodiments, an electromagnetic radiation source is used. In certain embodiments, the radiation source can deliver radiation at a desired dose to a desired site. In certain embodiments, the radiation source used can be a coherent or a non-coherent sources including, but not limited to, a laser, a lamp, a light, an optoelectric magnetic device, a diode.

In certain other embodiments, a radiation source can be used that is capable of directing radiation to a site of interest, for example, a laser with optical fiber delivery device, or a fiberoptic insert, or a lense used for interstitial or open field light delivery.

The usefulness of a radiation source can be determined using the animal model described in detail in the Examples Section below. Other animal models are known to the skilled artisan and are discussed in, for example, Bellnier et al., 1995, Photochemistry and Photobiology 62:896-905; Endrich et al., 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

See, also, U.S. Pat. Nos. 6,013,053; 6,011,563; 5,976,175; 5,971,918; 5,961,543; 5,944,748; 5,910,510; 5,849,027; 5,845,640; 5,835,648; 5,817,048; 5,798,523; 5,797,868; 5,793,781; 5,782,895; 5,707,401; 5,571,152; 5,533,508; 5,489,279; 5,441,531; 5,344,434; 5,219,346; 5,146,917; 5,054,867, which describe sources of radiation useful for the described methods.

Drugs that can be Delivered with the Described Methods

In accordance with the invention, any kind of molecule can be delivered using the described methods including, but not limited to, sugars, proteins, glycoproteins, phosphoproteins, nucleic acids, oligonucleotides, polynucleotides, oligonucleotides, RNA, DNA, modified nucleotides, modified polynucleotides, modified oligonucleotides, viral polynucleotides, vectors, plasmids (e.g., Bluescript, pUC, M13, etc.), lambda vectors, YAC vectors, lipids, lipoproteins, viruses, drugs, chemotherapeutics, hydrophilic molecules, polar molecules, hydrophobic molecules, charged molecules (e.g., ions), amphipathic molecules, encapsulated molecules.

In certain embodiments, the drug has a molecular weight from about 2 dalton to about 10 gigadalton, more preferably from about 20 dalton to about 5 gigadalton, more preferably from about 50 dalton to about 2.5 gigadalton, more preferably from about 100 dalton to about 1 gigadalton, more preferably from about 500 dalton to about 500 megadalton, more preferably from about 1 kilodalton to about 250 megadalton, more preferably from about 2.5 kilodalton to about 125 megadalton, more preferably from about 5 kilodalton to about 50 megadalton, more preferably from about 10 kilodalton to about 25 megadalton, more preferably from about 25 kilodalton to about 12.5 megadalton, more preferably from about 50 kilodalton to about 5 megadalton, more preferably from about 100 kilodalton to about 2.5 megadalton, more preferably from about 250 kilodalton to about 1 megadalton, and most preferably about 500 kilodalton.

In certain other embodiments, the drug has a molecular weight of at least about 50 kilodalton, more preferably at least about 100 kilodalton, more preferably at least about 250 kilodalton, more preferably at least about 500 kilodalton, more preferably at least about 1 megadalton, more preferably at least about 5 megadalton.

In certain embodiments, the drug includes, but is not limited to, peptides or proteins, hormones, analgesics, anti-migraine agents, anti-coagulant agents, anti-emetic agents, cardiovascular agents, anti-hypertensive agents, narcotic antagonists, chelating agents, anti-aniginal agents, chemotherapy agents, sedatives, anti-neoplastics, prostaglandins and antidiuretic agents, bradykinins, eicosanoids, histamines, osmolality modifiers such as mannitol.

In certain other embodiments, the drug includes, but is not limited to, peptides, proteins or hormones such as insulin, calcitonin, calcitonin gene regulating protein, somatropin, somatotropin, somatostatin, atrial natriuretic protein colony stimulating factor, betaseron, erythropoietin (EPO), luteinizing hormone release hormone (LHRH), tissue plasminogen activator (TPA), interferons such as .alpha., .beta. or .gamma. interferon, insulin-like growth factor (somatomedins), growth hormone releasing hormone (GHRH), oxytocin, estradiol, growth hormones, leuprolide acetate, factor VIII, interleukins such as interleukin-2, and analogues thereof; analgesics such as fentanyl, sufentanil, hydrocodone, oxymorphone, methodone, butorphanol, buprenorphine, levorphanol, diclofenac, naproxen, morphine, hydromorphone, lidocaine, bupivacaine, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondanesetron, domperidone, metoclopramide, and analogues thereof; cardiovascular agents, anti-hypertensive agents and vasodilators such as diltiazem, nifedipine, verapamil, clonidine, isosorbide-5-mononitrate, organic nitrates, agents used in the treatment of heart disorders, and analogues thereof; sedatives such as benzodiazepines, phenothiozines, and analogues thereof; narcotic antagonists such as naltrexone, naloxone, and analogues thereof; chelating agents such as deferoxamine, and analogues thereof; anti-diuretic agents such as desmopressin, vasopressin, and analogues thereof; anti-anginal agents such as nitroglycerine, and analogues thereof; anti-neoplastics such as 5-fluorouracil, bleomycin, and analogues thereof; prostaglandins and analogues thereof; and chemotherapy agents such as vincristine, and analogues thereof.

In certain other embodiments, the drug includes, but is not limited to, antiinfectives such as antibiotics and antiviral agents; analgesics and analgesic combinations; anorexics; antihelminthics; antiarthritics; hypnotics; immunosuppressives; muscle relaxants; parasympatholytics; antiasthmatic agents; antiparkinsonism drugs; antipruritics; antipsychotics; anticonvulsants; antidepressants; antidiabetic agents; antidiarrheals; antihistamines; antiinflammatory agents; antimigraine preparations; antinauseants; antineoplastics; antipyretics; antispasmodics; anticholinergics; sympathomimetics; xanthine derivatives; central nervous system stimulants; cough and cold preparations, including anti-histamine decongestants; cardiovascular preparations including calcium channel blockers, beta-blockers such as pindolol, antiarrhythmics, antihypertensives, diuretics, and vasodilators including general coronary, peripheral and cerebral; hormones such as the estrogens estradiol and progesterone and other steroids, including corticosteroids; psychostimulants; sedatives; tranquilizers, and analogs of any of the above.

See, also, U.S. Pat. Nos. 5,997,501; 5,993,435; 5,916,910; 5,980,948; 5,980,932, which describe drugs that can be delivered with the described methods.

In certain other embodiments, any drug (e.g., any compound, molecule, ion, or atom) can be delivered using the described methods and the best conditions for the delivery of a drug of interest can be determined using, for example, the hamster model described in the Examples Section below. Other animal models known in the art can also be used. Such animal models are described in, for example, Bellnier et al., 1995, Photochemistry and Photobiology 62:896-905; Endrich et al, 1980, Res. Exp. Med. 177:126-134; ten Tije et al., 1999, Photochem. Photobiol. 69:494-499; Abels et al., 1997, J. Photochem. Photobiol. B. 40:305-312; Fingar et al., 1992, Cancer Res. 52:4914-4921; Milstone et al., 1998, Microcirculation. 5:153-171; Kuhnle et al., 1998, J. Thorac. Cardiovasc. Surg. 115:937-944; Scalia et al., 1998, Arterioscler. Thromb. Vasc. Biol. 18:1093-1100; Iida et al., 1997, Anesthesiology 87:75-81; Dalla Via et al., 1999, J. Med. Chem. 42:4405-4413; Baccichetti, et al., 1992, Farmaco. 47:1529-1541; Roberts et al., 1989, Photochem. Photobiol. 49:431-438.

Timing for Introducing the Drug into the Organism for Delivery with the Described Methods

In certain embodiments, the drug may be introduced into the organism for delivery using the described methods before the photosensitizer is supplied to the organism and before radiation is employed in the described methods. In certain other embodiments, the drug may be introduced into the organism for delivery using the described methods after the photosensitizer is supplied to the organism but before the organism is irradiated. In certain other embodiments, the drug may be introduced into the organism for delivery using the described methods after the photosensitizer is supplied to the organism and after radiation. In certain other embodiments, the drug may be introduced into the organism for delivery using the described methods while the photosensitizer is supplied to the organism. In certain other embodiments, the drug may be introduced into the organism for delivery using the described methods while the organism is irradiated.

Dosage of the Drug Delivered using the Described Methods

In accordance with the invention, the drug is supplied to the organism of interest for delivery using the described methods at a dosage that is sufficient to allow the drug to be delivered at the desired site. For example, if the desired site for delivery of the drug is in the kidney, the liver, the brain, a muscle, the skin, or anywhere else in the organism, it is desirable to supply the drug to the organism at a dose that is sufficient for the drug to reach the site for delivery using the described methods.

Pharmacokinetic data on the distribution on drugs are well known in the art and a skilled artisan could readily determine a suitable dosage for the drug.

In certain preferred embodiments, a drug delivered with the described methods can be concentrated in a target tissue so that a smaller total amount per individual organism (e.g., per patient) is required to achieve a similar or identical therapeutic or diagnostic effect. This will result in lower toxicities and/or side effects for many therapeutic drugs including, but not limited to, chemotherapeutics, anti-infectives, anti-fungals.

In certain other embodiments, a drug is supplied to the organism for delivery using the described methods at a dose from about 0.5 microgram of drug per kilogram of body weight (i.e., the body weight of the organism or patient) (μg/kg) to about 10 milligram of drug per kilogram of body weight (mg/kg), more preferably from about 1 μg/kg to about 6 mg/kg, more preferably from about 2 μg/kg to about 3 mg/kg, more preferably from about 4 μg/kg to about 1.5 mg/kg, more preferably from about 8 μg/kg to about 0.75 mg/kg, more preferably from about 20 μg/kg to about 350 μg/kg, more preferably from about 40 μg/kg to about 200 μg/kg, more preferably from about 60 μg/kg to about 100 μg/kg, and most preferably about 80 μg/kg.

Animals, Tissues and Cells to which Drugs can be Delivered using the Described Methods

In certain embodiments, a drug may be delivered to an organism of any subspecies, species, genus, family, order, class, division, or kingdom. In a certain preferred embodiment, the organism is a human (a patient). In certain other embodiments, the organism is a mammal, a primate, a farm animal, a rodent, a bird, cattle, a cow, a mouse, a cat, a dog, a chimpanzee, a hamster, a fish, an ungulate, etc.

In certain embodiments, the drug may be delivered to any organ or tissue in the organism including, but not limited to, connective tissue, nervous tissue, muscle tissue, epithelia, adipose tissue, heart, liver, kidney, lung, pancreas, intestine, brain, sciatic nerve, spinal cord, thymus, glands, skeletal muscle, smooth muscle, prostate, uterus, stomach, bladder, etc.

In certain other embodiments, the drug may be delivered to any cell type in the organism of interest including, but not limited to, endothelial cells, fibroblasts, leukocytes, macrophages, lymphocytes, epithelial cells, cells of the immune system, muscle cells, neurons, glial cells, oligodendrocytes, Schwann cells, keratinocytes, hepatocytes, erythrocytes, platelets, etc.

In certain other embodiments, the drug may be delivered to cells that are, for example, proliferating, non-proliferating, differentiating, differentiated, migrating.

Diseases that can be Treated or Diagnosed using the Described Methods

In accordance with the invention, any condition in an organism of interest may be diagnosed and/or treated using the described methods.

In certain preferred embodiments, the described methods are usefull in many areas of therapeutic medicine where localized or enhanced drug delivery has been problematic including, but not limited to, solid tumor drug delivery, gene therapy, delivery of therapeutics to wound sites, or delivery of diagnostic reporter molecules (e.g., radionuclide labeled antibodies).

In certain embodiments, the conditions that may be diagnosed and/or treated using the disclosed methods include, but are not limited to, inflammatory and infectious diseases, such as, for example, septic shock, hemorrhagic shock, anaphylactic shock, toxic shock syndrome, ischemia, cerebral ischemia, administration of cytokines, overexpression of cytokines, ulcers, inflammatory bowel disease (e.g., ulcerative colitis or Crohn's disease), diabetes, arthritis, asthma, cirrhosis, allograft rejection, encephalomyelitis, meningitis, pancreatitis, peritonitis, vasculitis, lymphocytic choriomeningitis, glomerulonephritis, uveitis, ileitis, inflammation (e.g., liver inflammation, renal inflammation, and the like), bum, infection (including bacterial, viral, fungal and parasitic infections), hemodialysis, chronic fatigue syndrome, chronic pain, priapism, cystic fibrosis, stroke, cancers (e.g., breast, melanoma, carcinoma, and the like), cardiopulmonary bypass, ischemic/reperfusion injury, gastritis, adult respiratory distress syndrome, cachexia, myocarditis, autoimmune disorders, eczema, psoriasis, heart failure, heart disease, atherosclerosis, dermatitis, urticaria, systemic lupus erythematosus, Alzheimer's disease, Parkinson's disease, multiple sclerosis, AIDS, AIDS dementia, chronic neurodegenerative disease, amyotrophic lateral sclerosis, schizophrenia, depression, premenstrual syndrome, anxiety, addiction, migraine, Huntington's disease, epilepsy, neurodegenerative disorders, gastrointestinal motility disorders, obesity, hyperphagia, solid tumors (e.g., neuroblastoma), malaria, hematologic cancers, myelofibrosis, lung injury, graft-versus-host disease, head injury, CNS trauma, hepatitis, renal failure, liver disease (e.g., chronic hepatitis C), drug-induced lung injury (e.g., paraquat), myasthenia gravis (MG), ophthahnic diseases, post-angioplasty, restenosis, angina, coronary artery disease, treatment of intimal hyperplasia, prevention of restenosis post angioplasty; prevention of restenosis post vascular graft procedures; prevention of restenosis post arteriovenous fistula; treatment of intimal hyperplasia post vascular grafts; treatment of intimal hyperplasia after angioplasty; treatment of intimal hyperplasia in stented vessels (in-stent restenosis); port-wine stain and other hemangiomas; arteriovenous malformations and aneurysms, diabetic maculopathy/retinopathy, glaucoma.

### EXAMPLES

The following examples are provided to illustrate the methods of the invention and should not be considered to limit the invention.

### EXAMPLE I

Materials And Methods

Window Chamber Implantation

Syrian golden hamsters (Charles River Laboratories, Kingston, N.Y.) weighing between 60-70 gram were surgically implanted with titanium back-pack window chambers as described (Endrich et al., 1980; Colantuoni et al., 1984; Friesenecker et al., 1994). Prior to the surgical procedure, the dorsal surface of the mouse was shaved with electric clippers (Sunbeam Oster 2-Speed, 150 Cadillac Lane, McMinnville, Tenn., 37110) and then the shaved skin covered in a depilatory cream (Nair, Carter Products, New York, N.Y., 10105) for 10 minutes to remove the remaining hair. A dorsal skin fold consisting of two layers of skin and muscle tissue was then sandwiched between two opposing titanium frames (Campus Research Machine Shop, University of California, San Diego 9500 Gilman Drive, La Jolla, Calif.) with a 15 mm circular opening in each. Layers of skin and muscle fascia were separated from the sub-cutaneous tissue, and removed until a thin monolayer of muscle and one layer of intact skin remained. A coverglass (Type Circle 1, Part #12-545-80 sourced from Fisher Scientific, 2761 Walnut Avenue, Tustin, Calif., 92780) held by an expansion ring in the circular window of one titanium frame was then placed on the exposed tissue to allow direct microscopic visualization of the vasculature. The window in the second opposing titanium frame was left open exposing the intact skin. Two days following the implantation of the titanium chamber, an in-dwelling PE10 catheter (VVR Scientific, Westchester, Pa.) was implanted in the carotid artery. The catheter tubing was passed sub-cutaneously from the ventral to the dorsal side of the neck, and exteriorized through the skin at the base of the chamber. The patency of the catheter was ensured by daily flushing of the in-dwelling implanted tip with 0.005-0.01 ml of heparinized saline (40 IU/ml). The heparin was sourced from Upjohn Co., 100 Route 206N, Prepack, N.J., 07977, and the saline from Abbott Laboratories, North Chicago, Ill., 60064.

Microvascular observations using an intra-vital microscope were not undertaken until at least 4 days post-chamber implantation to mitigate against post-surgical trauma, and to confirm that blood vessels within the chamber were functioning and intact and patent. A chamber was considered suitable for subsequent studies if microscopic examination of the preparation met the following criteria (as applied in Friesenecker et al., 1994):

1. there were no signs of bleeding and /or edema within the chamber;

2. the systemic mean blood pressure of the animal was greater than 80 mm Hg;

3. the heart rate of the animal was greater than 320 beats per minute as measured by a Beckman recorder R611 (Beckman Coulter, 4300 N. Harbour Boulevard, Fullerton, Calif., 92634) with a Spectramed DTx pressure transducer (Model TNF-R, Viggo-Spectramed);

4. the systemic hematocrit was greater than 45% (Becton Readacrit centrifuge; Becton Dickinson, 1 Becton Crive, Franklin Lakes, N.J., 07917);

5. the number of immobilized leukocytes and those flowing with venular endothelial contact in the chamber was less than 10% of all passing leukocytes at a time point control within the chamber;

6. there was no evidence of post-surgical infection in the chamber or surrounding tissue.

Intra-Vital Microscope and Methods

The intra-vital microscopic studies were undertaken on un-sedated animals held in a Plexiglass tube (Campus Research Machine Shop, University of California, San Diego 9500 Gilman Drive, La Jolla, Calif.) from which the window chamber sandwich protruded horizontally, allowing visualization of the chamber on the microscope stage. The Plexiglass tube acted to restrain the animals without impeding respiration. The intra-vital microscopy was performed using a Leitz Ortholux II (McBain Instruments, Inc., 9601 Variel Avenue, Chatsworth, Calif., 91311) fitted with a Leitz Wetzlar 25× saline immersion objective lens, 0.6 numerical aperture (McBain Instruments, Inc., 9601 Variel Avenue, Chatsworth, Calif., 91311), a Leitz Wetzlar 10× dry Planfluotar lens, 0.3 numerical aperture (McBain Instruments, Inc., 9601 Variel Avenue, Chatsworth, Calif., 91311) and a Leitz Wetzlar 4× EF dry lens, 0.12 numerical aperture (McBain Instruments, Inc., 9601 Variel Avenue, Chatsworth, Calif., 91311). A 100 Watt Hg light source (Olympus Corporation, 2 Corporate Center Drive, Melville, N.Y., 11747-3157) was used for both trans- and epi-illumination. For the trans-illumination studies the light was filtered using a 420 nm blue filter which selectively passed light in the region of the maximum absorbance band of hemoglobin, causing the red blood cells to appear as dark objects against a gray background. In addition, a heat filter was placed in the light path prior to the condenser to prevent hyperthermic effects on the tissue being examined. For the fluorescence epi-illumination studies, the microscope was also fitted with a Leitz Ploemopak system (McBain Instruments, Inc., 9601 Variel Avenue, Chatsworth, Calif., 91311). For visualization of the fluorescein diisothiocyanate dextran (FITC-Dextran) conjugate (Sigma Scientific, PO Box 14508, St. Louis, Mo., 63178) the Ploempak I₃ cube (McBain Instruments, Inc., 9601 Variel Avenue, Chatsworth, Calif., 91311) with spectral characteristics of 450-490 nm excitation, 520 nm emission was used.

The intra-vital microscopic images were viewed by a closed circuit video system, consisting of a video cassette recorder and monitor (Sony PVM 1271Q, Sony Corporation, 680 Kinderkamack Rd., Oradell, N.J., 07649 and a silicon-intensified camera (sensitivity 7×10⁻³ foot candles; Cohu, Inc., PO Box 85623, San Diego, Calif., 92186) and were recorded onto standard 180 min video cassette tapes. The functional capillary density (FCD) in microscopic fields within the window chamber was determined as previously described, whereby a capillary was defined as functional if red blood cells (RBCs) passed through the length of capillary within a 45 second observation period. The FCD was defined as the number of capillaries in which RBCs passed which were present in 5-10 laterally adjacent fields of view. The arteriolar and venular diameters were determined pre-, during and post-PhotoPoint™ therapy using a previously published live optical image shearing technique using an image-shearing system (Digital Video Image Shearing Monitor, Model 908, IPM, San Diego, Calif.).

PhotoPoint™ Therapy of Hamsters Bearing Dorsal Window Chambers

Following the post-surgical care and observation period (at least 4 days), hamsters bearing a dorsal window chamber were placed in the Plexiglass restrainer on the microscope stage, and then injected with either the photosensitizer MRV6401, which is indium methyl pyropheophorbide iravant Medical Technologies, 336 Bollay Drive, Santa Barbara, Calif., 93117), formulated in egg yolk phospholipid (Avanti Polar Lipids, Inc., 700 Industrial park Avenue, Alabaster, Ala., 35007) and diluted in solution of 5% dextrose : water (Abbott Laboratories, N. Chicago, Ill., 60064) or the photosensitizer SiET2 (Miravant Medical Technologies, 336 Bollay Drive, Santa Barbara, Calif., 93117). Both photosensitizers were administered via the intra-carotid (i.c.) catheter to a final dose of either 0.05 mg/kg body weight or 0.15 mg/kg body weight for MRV6401, or 1.0 mg/kg body weight for SnET2. The time taken to administer either drug via a slow i.c. push was approximately 2 min, and was followed by a flush of 0.1 ml heparin-saline (a total of 15.4 Units heparin)). The heparin was sourced from Upjohn Co., 100 Route 206N, Prepack, N.J., 07977, and the saline from Abbott Laboratories, North Chicago, Ill., 60064.

Ten minutes after the completion of the heparin-saline flush, the tissue in the window chamber was exposed to filtered light from the mercury trans-illumination source that activated the drug. In contrast to the power used for standard visualization of the tissue (approximately 0.3 mW/cm²), the power output from either the photo-activating mercury light source or red diode laser (Miravant DD4—output wavelength 665 nm; Miravant Medical Technologies, 336 Bollay Drive, Santa Barbara, Calif., 93117) was increased for the duration of the photo-activation period to achieve a higher power density. The activation beam from the mercury source was filtered with a 1 mm thick BG25 filter (Schott Glass Technologies, Inc., 400 York Avenue, Duryea, Pa., 18642), which delivered 425 nm light at the increased power density of between 21-100 mW/cm², resulting in a total energy dose of between 10-50 J/cm². The activation beam from the laser was directed through the condenser lens of the microscope via a 400 μm inner core optical fiber fitted with a microlens at the delivery end (Miravant, Model ML 1-0400-EC, Miravant Medical Technologies, 336 Bollay Drive, Santa Barbara, Calif., 93117) enabling projection of red 665 nm light onto the window chamber. All measurements of the power of either the blue or red light incident upon the window chamber were made using an Ophir Optronics Nova Display power meter (Serial number 45855) fitted with an Ophir PD 300 filtered detector head (Serial number 35211) from Ophir Optronics, Inc., 9 Electronics Avenue, Danvers Industrial Park, Danvers, Mass., 01923. This power meter allowed precise power output measurements to be made at specific wavelengths, in this case 420 nm, 425 nm and 665 nm. In addition, the power density distribution across the illumination field was determined using an isodosimetry detector probe (Miravant DP 10208—Miravant Medical Technologies, 336 Bollay Drive, Santa Barbara, Calif., 93117) consisting of 200 μm inner core optical fiber with a spherical diffusing tip (0.8 mm diameter). The probe tip was passed across the field, and the evenness of illumination determined by measuring the light power transmitted from the tip through the optical fibre to the Ophir Optronics Nova Display power meter (serial number 45855)fitted with an Ophir PD 300 filtered detector head (serial number 35211) both from Ophir Optronics, Inc., 9 Electronics Avenue, Danvers Industrial Park, Danvers, Mass., 01923.

For the duration of the photodynamic activation, the 420 nm blue filter (described above) was removed. The tissue being treated was visually monitored throughout the procedure and the real-time images recorded to video-tape. At the conclusion of the activation period, the power output from the mercury source was reduced and the BG25 filter removed and replaced with the 420 nm blue filter for on-going monitoring of the tissue.

Determination of Vessel Permeability Using FITC-Dextran

The permeability of vessels pre- and post-PhotoPoint™ therapy in (a) treated with photosensitizer and light and (b) light only and (c) drug only control animals was determined using epi-fluorescence visualization of the vascular leakage of a conjugate of FITC-Dextran of 150 kD molecular weight (Sigma Scientific, PO Box 14508, St. Louis, Mo., 63178). The FITC-Dextran was administered via the carotid catheter, and the treatment field in the window chamber examined using the epi-fluorescence equipment and settings described in (ii) above. Typically, 0.15-0.25 ml of a 5% w./vol. solution of FITC-Dextran in isotonic saline was administered via i.c. push over 1.5 min, followed by a heparin-saline flush of 0.1 ml (15.4 Units heparin).

The distribution of the fluorescence emitted from the FITC-Dextran was then monitored over a period of between 0.5-1 hour, and also was monitored for a further 0.5-1 hr approximately 24 hours later to determine if fluorescence could still be detected in the vasculature or in the tissue following extravasation.

Results

A total of 10 hamsters bearing dorsal window chambers were utilized in this study. Of these, 9 were evaluable, with the 10^{th} hamster suffering a hyperthermic injury within the treatment field caused by intense illumination of focused filtered blue light from the mercury source prior to drug administration. This animal was therefore not administered drug and was withdrawn from the study. The treatment protocols and parameters for all 10 hamsters are shown in Table 1.

Interesting were the results obtained for hamsters A33, A35, A41, A45, A46, A69 and A70. In each of these animals, the degree of vascular permeability induced by the photodynamic process was determined by examining the extravasation of FITC-Dextran (150 kD) from the vasculature into the surrounding tissue. The FITC-Dextran conjugate was administered at times varying from 30 to 90 min following the completion of control light illumination alone, or PhotoPoint™ therapy in hamsters A35, A41 and A46 (sensitized with MRV6401), hamsters A69 and A70 (sensitized with SnET2) and hamsters A33 and A45 (control animals administered the drug vehicle only followed by light exposure). The results of the FITC-Dextran analysis are described in Table 2.

Discussion

When PhotoPoint™ therapy was administered using the parameters described in Table 1, it mediated a number of post-treatment events. These events can be summarized as follows. Focal constrictions were apparent in arterioles and arteries within 30 sec following the commencement of PhotoPoint™ therapy mediated by both MV6401 and SnET2. Dilation was apparent in venules and veins within 30 sec following the commencement of PhotoPoint™ therapy mediated by MV6401, however some minor constriction was noted in venules and veins during PhotoPoint™ therapy mediated by SnET2. There was a rapid (within 40 sec) initial loss of capillary flow using both drugs, but destruction of the capillaries was not evident, either immediately or 24 hrs after PhotoPoint™ therapy. There appeared to be rapid thrombus formation in some arterioles and post-capillary venules following PhotoPoint™ therapy mediated by both drugs. Leukocyte adhesion to blood vessel walls was apparent in post-capillary venules. Leukocyte invasion into the tissue of the chamber was apparent at time points of 24 hrs and longer following PhotoPoint™ therapy. FITC-Dextran extravasation from blood vessels, and subsequent retention in tissue within the chamber was mediated by PhotoPoint™ therapy. This was indicative of increased permeability of vessels walls induced by PhotoPoint™. In control animals that received either light or drug alone, no extravasation of the FITC-Dextran was apparent, and there was no evidence of FITC-Dextran retention within the tissues in the chamber. Whilst resulting in severe damage to the entire irradiation field, as evidenced by apparent thrombus formation and loss of capillary flow, the dosimetry of photosensitizer and light used in this experiment still facilitated enhanced delivery of the FITC-Dextran to the surrounding tissue. In some clinical situations such damage may be desirable. However, to determine if delivery of the FITC-Dextran could be enhanced without severe damage to the irradiation field, the following experiments were undertaken.

### EXAMPLE 2

Materials And Methods

Window Chamber Implantation

Male mice of strain C3H (sourced from The Jackson Laboratory, 600 Main Street, Bar Harbor, Me. 04609 USA) weighing between 28-30 gram were surgically implanted with titanium back-pack window chambers in a similar manner to that described for Syrian Golden hamsters as described above. Prior to the surgical procedure, the dorsal surface of the mouse was shaved with electric clippers (Sunbeam Oster 2-Speed, 150 Cadillac Lane, McMinnville, Tenn., 37110) and then the shaved skin covered in a depilatory cream (Nair, Carter Products, New York, N.Y., 10105) for 10 minutes to remove the remaining hair. Then a dorsal skin fold consisting of two layers of skin and muscle tissue was sandwiched between two opposing titanium frames (Campus Research Machine Shop, University of California, San Diego 9500 Gilman Drive, La Jolla, Calif.) with a 14 mm circular opening in each. Layers of skin and muscle fascia were separated from the sub-cutaneous tissue, and removed until a thin monolayer of muscle and one layer of intact skin remained. A coverglass (Type Circle 1, Part #12-545-80 sourced from Fisher Scientific, 2761 Walnut Avenue, Tustin, Calif., 92780) held by an expansion ring in the circular window of one titanium frame was then placed on the exposed tissue to allow direct microscopic visualization of the vasculature. The window in the second opposing titanium frame was left open exposing the intact skin.

Microvascular observations using an intra-vital microscope were not undertaken until at least 2 days post-chamber implantation to mitigate against post-surgical trauma, and to confirm that blood vessels within the chamber were functioning and intact and patent. A chamber was considered suitable for subsequent studies if microscopic examination of the preparation met the following criteria (as applied in Friesenecker et al., 1994):

1. there were no signs of bleeding and /or edema within the chamber;

2. there was minimal fascial tissue remaining following the surgery

3. there was no evidence of post-surgical infection in the chamber or surrounding tissue

Intra-Vital Microscope and Methods

The intra-vital microscopic studies were undertaken on un-sedated animals held in a Plexiglass tube (manufactured by Miravant Medical Technologies, Inc., 336 Bollay Drive, Santa Barbara, 93117) from which the window chamber sandwich protruded horizontally, allowing visualization of the chamber on the microscope stage. The Plexiglass tube acted to restrain the animals without impeding respiration. The intra-vital microscopy was performed using a Leitz Dialux 22 (West LA Microscope Co., Butler Avenue, Santa Monica, 90025) fitted with a Leitz Wetzlar 20× L20 lens (0.32 numerical aperture), a Leitz Wetzlar 10× Planfluotar lens (0.30 numerical aperture), a Leitz Wetzlar 4× EF lens (0.12 numerical aperture), a Leitz Wetzlar 2.5× P1 lens (0.08 numerical aperture, and an Olympus 20× Wplan water immersion lens (0.4 numerical aperture). The intra-vital microscope system used for these studies was fitted with two trans-illumination light sources and one epi-illumination light source. The two trans-illumination sources were used in the following manner. One trans-illumination light source was used for imaging the tissue within the window chamber, and the other was used as the irradiation source for activating the photosensitizer in the tissue. The imaging source was a 100 mWatt mercury arc lamp (Type 307-143.004 from Ernst Leitz Wetzlar GmBH, Germany) which was powered by an HBO 100 power supply (LEP Ltd., Scarsdale, N.Y.). The output from this source was filtered using a #H43157 interference filter (Edmund Scientific, 101 East Gloucester Pike, Barrington, N.J., 08007-1380), to produce a beam of 410 nm light. The irradiation source was a Model DD4 Diode Laser (Miravant Medical Technologies 336 Bollay Drive., Santa Barbara, Calif., 93117), which produced 664 nm light.

The beams from the two light sources were combined using a 25 mm beam splitting cube (Part #H45201, Edmund Scientific, 101 East Gloucester Pike, Barrington, N.J., 08007-1380), The treatment light was delivered from the laser via a 400 nm optical fiber (Miravant Medical Technologies, Inc., 336 Bollay Drive, Santa Barbara, 93117) which was coupled to the beam splitting cube by means of a standard SMA-905 fiberoptic connector attached to one face of the cube. The fiberoptic connector and beam splitting cube were mounted and positioned on the underside of the registration stage, above the microscope condenser lens in the center of the standard trans-illumination light path. Thus the imaging beam and the activating irradiation beam could be combined and directed evenly onto the tissue surface within the window chamber. Typically, the power density of the imaging light was less than 0.6 mWatt/cm². All measurements of the power of either the blue imaging or red activating light incident upon the window chamber were made using an Ophir Optronics Nova Display power meter (Serial number 45855) fitted with an Ophir PD 300 filtered detector head (Serial number 35211) from Ophir Optronics, Inc., 9 Electronics Avenue, Danvers Industrial Park, Danvers, Mass., 01923. This power meter allowed precise power output measurements to be made at specific wavelengths, in this case 420 nm, 425 nm and 665 nm. In addition, the power density distribution across the illumination field was determined using an isodosimetry detector probe (Miravanit DP1 0208—Miravant Medical Technologies, 336 Bollay Drive, Santa Barbara, Calif., 93117) consisting of 200 μm inner core optical fiber with a spherical diffusing tip (0.8 mm diameter). The probe tip was passed across the field, and the eveness of illumination determined by measuring the light power transmitted from the tip through the optical fibre to the Ophir Optronics Nova Display power meter (serial number 45855)fitted with an Ophir PD 300 filtered detector head (serial number 35211) both from Ophir Optronics, Inc., 9 Electronics Avenue, Danvers Industrial Park, Danvers, Mass. 01923. The output power of the 664 nm activating light from the DD4 laser was adjusted so that the power density was 50 mW/cm² at the treatment site.

The Plexiglass restrainer was a custom built design. Briefly, it consisted of an acrylic tube of Plexiglass of the appropriate diameter (2.9 cm internal diameter) to comfortably, yet securely contain the mouse. The test mouse, with window chamber implanted, was held within the Plexiglass restrainer device that had holes down its length to provide for adequate air and ventilation. The mouse was held horizontal (lying on its side) within the restrainer, with the implanted titanium window chamber protruding outside the acrylic tube in a horizontal plane via a slot cut down the length of the tube. The acrylic tube was mounted on a pair of square end flanges 4 cm×4 cm which provided a flat base to prevent the tube from rolling. These flanges registered into slots on the registration stage of the microscope, and each flange had a protruding ear, which locked into a spring-loaded mechanism on the registration stage. This allowed the restrainer to be quickly mounted to the registration stage of the microscope in a repeatable position, and just as quickly removed. The registration stage consisted of a platen that attached to the top of the microscope viewing stage. The XY positioning mechanism of the microscope thus allowed the mouse under examination to be accurately and repeatable positioned under the appropriate objective lens for microscopic viewing of the vascular structures within the tissue in the window chamber. Even distribution of the imaging and activation light across the treatment field in the window chamber was achieved by means of a custom diffusing lens made by bonding two pieces of Roscolux 116 diffuser paper (Rosco Ltd., 112 N. Citrus Ave., Hollywood, Calif., 90038) to each side of a #H02105 optical window lens (Edmund Scientific, 101 East Gloucester Pike, Barrington, N.J., 08007-1380). This diffusing lens was attached to the side of the Plexiglass restrainer so that the trans-illumination light passed through it prior to reaching the treatment field within the window chamber.

After the combined activating and imaging light passed through the tissue and into the microscope light path via the objective lens, the activating light was removed by means of a 586ESP filter (Omega Optical Co., Brattleboro, Vt., 05302-0573) placed immediately in front of the CCD camera (Panasonic WV-BP334, Panasonic Corporation, Secaucus, N.J.), which was attached to the camera mount of the microscope.

For epi-illumination studies of FITC-Dextran fluorescence, the light source used was a 100 mWatt mercury arc lamp (Type 307-143.004 from Ernst Leitz Wetzlar GmBH, Germany) which was powered by an HBO 100 power supply (LEP Ltd., Scarsdale, N.Y.). This light source was attached to the epi-illumination port of the microscope.

All lenses were sourced from McBain Instruments, Inc., 9601 Variel Avenue, Chatsworth, Calif., 91311. A Leica 100 Watt Hg light source (McBain Instruments, Inc., 9601 Variel Avenue, Chatsworth, Calif., 91311) was used for both trans- and epi-illumination. For the trans-illumination studies the light was filtered using a 405 nm blue filter which selectively passed light in the region of the maximum absorbance band of hemoglobin, causing the red blood cells to appear as dark objects against a gray background. In addition, a heat filter was placed in the light path prior to the condenser to prevent hyperthermic effects on the tissue being examined. For the fluorescence epi-illumination studies, the microscope was also fitted with a Leitz Ploemopak system (McBain Instruments, Inc., 9601 Variel Avenue, Chatsworth, Calif., 91311). For visualization of the fluorescein diisothiocyanate dextran (FITC-Dextran) conjugate (Sigma Scientific, PO Box 14508, St. Louis, Mo., 63178) the Ploempak I₃ cube (McBain Instruments, Inc., 9601 Variel Avenue, Chatsworth, Calif., 91311) with spectral characteristics of 450-490 nm excitation, 520 nm emission was used.

The intra-vital microscopic images were viewed by a closed circuit video system, consisting of a video cassette recorder (JVC Model HR-S4600U, JVC Corporation, Wayne, N.J., 07470) and monitor (Sony Trinitron PVM 14N2V, Sony Corporation, 680 Kinderkamack Rd., Oradell, N.J., 07649) and a CCD camera (sensitivity 7×10⁻³ foot candles; Panasonic W-BP34, Panasonic Corporation, Secaucus, N.J.) and were recorded onto standard 180 min VHS video cassette tapes. The functional capillary density in microscopic fields within the window chamber was determined as previously described, whereby a capillary was defined as functional if red blood cells (RBCs) passed through the length of capillary within a 45 second observation period. The FCD was defined as the number of capillaries in which RBCs passed which were present in 5-10 laterally adjacent fields of view.

PhotoPoint™ Therapy of Mice Bearing Dorsal Window Chambers

Following the post-surgical care and observation period, mice bearing a dorsal window chamber were placed in the Plexiglass restrainer on the microscope stage, and then injected with either the photosensitizer MRV6401(Miravant Medical Technologies, 336 Bollay Drive, Santa Barbara, Calif., 93117) formulated in egg yolk phospholipid (Avanti Polar Lipids, Inc., 700 Industrial Park Avenue, Alabaster, Ala., 35007) or egg yolk phospholipid as a control. The photosensitizer or vehicle control solutions were administered via the intra-venous (i.v.) tail vein route, with the photosensitizer being administered to a final dose of 0.05 mg/kg body weight. The time taken to administer either drug or vehicle control solution via a slow i.v. push was approximately 1 min. Ten minutes after the completion of the administration, the tissue in the window chamber was exposed to filtered light from the trans-illumination imaging source and the laser activating source. In contrast to the power used for standard visualization of the tissue (approximately 0.3 mW/cm² of blue light), the power output from the diode laser (Miravant DD4—output wavelength 665 nm) increased for the duration of the photo-activation period to achieve a higher power density of 50 mW/cm² of red activating light. Total doses of red light administered to the animals were as described in Table 3.

Determination of Vessel Permeability Using FITC-Dextran

The permeability of vessels pre- and post-PhotoPoint™ therapy in (a) treated and (b) light and (c) drug only control animals was determined using epi-fluorescence visualization of the vascular leakage of a conjugate of FITC-Dextran of 150 kD molecular weight (Sigma Scientific, PO Box 14508, St. Louis, Mo., 63178). The FITC-Dextran was administered via the i.v. tail vein route, and the treatment field in the window chamber examined using the epi-fluorescence equipment and settings described in (ii) above. Typically, 0.15-0.25 ml of a 5% w./vol. solution of FITC-Dextran in isotonic saline was administered. The distribution of the fluorescence emitted from the FITC-Dextran was then monitored over a period of between 0.5-1 hour, and also was also monitored at later times (as described in Table 4) to determine if fluorescence could still be detected in the vasculature or in the tissue following extravasation.

Results

A total of four mice bearing dorsal window chambers were utilized in this study. All were evaluable. The summary of the treatment parameters for these animals is shown in Table 3. Animals #1 and #4 were control animals. Animal #1 received no photosensitizer and no activating light, but did receive the imaging light. Animal #4 received a 0.16 ml administration of the egg yolk phospholipid vehicle by slow push via the i.v. tail vein route, 10 min prior to activating light illumination. The test animals #2 and #3 both received 0.05 mg MV6401/kg body weight formulated in an egg yolk phospholipid: 5% dextrose/water mixture (1 part egg yolk phospholipid: 80 parts 5% dextrose/water). A total volume of 0.16 ml of the MV6401/egg yolk phospholipid/5% dextrose/water mixture was delivered to each animal by slow push via the i.v. tail vein route 10 min prior to the commencement of the PhotoPoint™ therapy.

Discussion

These data in Tables 3 and 4 provide additional evidence to that presented above. In the experiments described in Tables 3 and 4 (above), the selective delivery of FITC-Dextran to the tissue was achieved using lower doses of PhotoPoint™ therapy than those used in the experiments described above. In contrast to the earlier experiments, the PhotoPoint™-mediated delivery of FITC-Dextran into the tissue was achieved without causing obvious damage to the tissue or vasculature (see Animal #2), and with some minimal damage in Animal #3. In both these animals, the majority of vessels appeared patent and were flowing, as evidenced by the flow of the FITC-Dextran seen in the plasma in all vessels in the chamber immediately after the fluorescent probe was administered. Subsequent observation showed the gradual extravasation of the FITC-Dextran into the tissue. Taken together, these data from the two experiments (Tables 1-4) confirm there is a dosage effect. Animals administered the same photosensitizer dose, but with higher light doses (greater than 20 J/cm²) exhibited a rapid and, in some cases permanent closure of arterioles, with a permanent cessation of capillary flow. In these high dose animals there was evidence of severe tissue damage (edema, cellular infiltrate) which progressed following PhotoPoint™ treatment. However, in the lower dose animals (those receiving 20 J/cm² or less of light) there was no evidence of widespread tissue or vascular damage. However, like the high dose animals, there was still a pronounced vascular leakage of FITC-Dextran into the surrounding tissue in these low dose animals. At the light doses used in the evaluable animals described in Tables 1-4, the leakage of FITC-Dextran only occurred when the photosensitizer (either MV6401 or SnET2) was present. The dosage effect described here supports the theory of the reciprocity of drug and light dosimetry in mediating a biological effect. However, it is probable that the selective delivery of drugs from the vasculature to the tissue may be mediated by even lower doses of drug and light than those described in these experiments. This reduced dosimetry may thus mediate the desired effect (i.e., selective local drug delivery) with sparing of all tissue and vascular structures in the treatment field.

Conclusions

The data presented in Examples 1 and 2 show that administering light after photosensitizer drug administration can induce selective structural and permeability changes in vascular structures. These changes are very rapid, and result in damage to arterioles and venules, with destruction of surrounding tissue occurring on a longer time scale as a consequence of infarction of the tissue.

The induced permeability changes in the vasculature have been shown to result in enhanced release of a macromolecule, in this case a 150 kD FITC-Dextran conjugate. Following PhotoPoint™-induced release from the vasculature, fluorescence microscopy revealed the molecule was retained in the treated tissue field for at least 48 hrs. This finding suggests that photodynamic treatment can be utilized to enhance local delivery of a variety of large and small molecular weight therapeutics to a treatment field.

Using these drug and light dosimetry combinations in the above Example, there was no wide-spread vessel destruction and loss of blood flow, as fluorescently tagged marker molecules (in this case a FITC-Dextran conjugate) could be administered systemically and still be observed within several seconds to be entering the target field via the still intact blood vessels. The FITC-Dextran was then seen to leak from these vessels in the target field over a period of several hours. Subsequently (during an observation 24 hrs later) it was noted that some vessels in the field were thrombosed, and some were destroyed. However, the FITC-Dextran, which had leaked from the vessels in the immediate post-PDT period, was still observed in the target field, but was not evident in the vessels or vessel remnants.

The observation that some vessels in the field were destroyed or thrombosed suggests that modifications of drug and light doses can achieve varying end results, depending on the therapeutic intention. For example, if the goal is to facilitate drug delivery, and also to achieve some long term destruction of the target tissue (e.g., in a tumor), then the dosimetry used in the experiments described in the above Example would be satisfactory. However, if the goal is to purely facilitate drug delivery, with minimal subsequent damage to the tissue (e.g., in a wound where the delivered therapeutic may enhance healing, or in a wound where the aim may be to deliver an antibiotic to combat or prevent infection), then the dosimetry of drug and light can be modified to increase vascular extravasation of an agent, but not be so severe as to result in undesired photodynamic tissue destruction.

### EXAMPLE 3

Materials and Methods

Window Chamber Implantation

Male mice of strain C3H (sourced from The Jackson Laboratory, 600 Main Street, Bar Harbor, Me. 04609, USA) weighing between 28-30 grams were surgically implanted with titanium back-pack window chambers in a similar manner to that described in Example 2. A chamber was considered suitable for subsequent studies if microscopic examination of the preparation met the same criteria as those described in Example 2.

Intra-Vital Microscopy and Methods

The intra-vital microscopic studies were undertaken on unsedated animals held in the same Plexiglass tube assembly as that described in Example 2. The intra-vital microscopy was also performed using the same Leitz Dialux 22 microscope fitted with the same objective lenses, and mercury lamp trans- and epi-illumination sources. However, for the studies described in Example 3, the irradiation source was not a DD4 laser, but rather, activation of the photosensitizers was undertaken using narrow band filtered light from the mercury lamp epi-illumination source. Two different wavelength bands were used to activate the photosensitizers, namely green or red light (see Table 5 below). These wavelengths were obtained by use of filter cubes placed in a Leitz Ploemopak illumination system (NcBain Instruments, Inc., 9601 Variel Avenue, Chatsworth, Calif., 91311, USA) fitted to the Leitz Dialux 22 microscope. For activation using green light epi-illumination (530 nm-560 nm) the BP 530-560 band pass excitation filter of the Leitz Ploemopak N2 cube (Cat. No. 513-609, E. Leitz Inc., Rockleigh, N.J., 07647, USA) was used. For activation using red light epi-illumination (660 nm-680 nm) a 670DF20 band pass filter (Cat. No., XF1028, Omega Optical Co., Brattleboro, Vt., 05302, USA) fitted in a Leitz Ploemopak cube was used.

In all cases, the activation of the photosensitizer was performed in a defined region that was smaller than the total field of tissue contained within the window chamber. The total mouse dorsal tissue contained within the window chambers was a circle of 1.0 cm diameter, corresponding to a total area of approximately 0.785 cm². The illumination field of the activating light was a circle of 0.225 cm diameter, corresponding to a total area of approximately 0.040 cm². Thus, approximately 5% of the total area of the chamber was directly illuminated. The power density and total energy doses of the respective wavelengths of activation are shown in Table 5.

The power density distribution across the illumination field was determined using an isodosimetry detector probe (Miravant DPI 0208—Miravant Medical Technologies, 336 Bollay Drive, Santa Barbara, Calif., 93117) consisting of a 200 micrometer inner core optical fiber with a spherical diffusing tip (0.8 mm diameter). The probe tip was passed across the field, and the evenness of illumination determined by measuring the light power transmitted from the tip through the optical fibre to the Ophir Optronics Nova Display power meter (Serial Number 45855). This power meter was fitted with an Ophir PD 300 filtered detector head (Serial Number 35211; Ophir Optronics, Inc., 9 Electronics Avenue, Danvers Industrial Park, Danvers, Mass., 01923).

The intra-vital microscopic images were viewed by a closed circuit video system and recorded onto standard 180 minute VHS video cassette tapes as described in Example 2.

Photosensitizer Administration and Activation

Mice bearing a dorsal window chamber were placed in the Plexiglass restrainer on the microscope stage for pre-treatment evaluation of the vascular structures. Prior to photosensitizer administration, the architecture of the vascular structures in the entire window chamber in all mice was examined using 410 nm filtered blue light from the mercury trans-illumination source of the intra-vital microscope, and the images which were generated were recorded on video tape for subsequent evaluation. The methods for imaging and recording were as described in Example 2. Briefly, the imaging source was a 100 mWatt mercury arc lamp (Type 307-143.004 from Ernst Leitz Wetzlar GmBH, Germany) which was powered by an HBO 100 power supply (LEP Ltd., Scarsdale, N.Y.). The output from this source was filtered using a #H43157 interference filter (Edmund Scientific, 101 East Gloucester Pike, Barrington, N.J., 08007-1380), to produce a beam of 410 nm light. Typically, the power density of the imaging light was less than 0.6 mWatt/cm². Two or three fields in each chamber were designated as fields of interest and their location recorded for post-treatment evaluation. These fields were chosen such that they were not adjacent to each other, and one of these fields was chosen so that it was within the region of the chamber that was to receive the activating light illumination.

Following identification, recording and designation of the fields of interest, mice were injected with either the photosensitizer MRV6401 (iravant Medical Technologies, 336 Bollay Drive, Santa Barbara, Calif., 93117) formulated in egg yolk phospholipid (Avalnti Polar Lipids, Inc., 700 Industrial Park Avenue, Alabaster, Ala., 35007), the photosensitizer SnET2 (Miravant Medical Technologies, 336 Bollay Drive, Santa Barbara, Calif., 93117), or egg yolk phospholipid as a vehicle control. The photosensitizers and vehicle control solutions were administered via the intra-venous (i.v.) tail vein route, with MV6401 being administered to a final dose of 0.05 mg/kg body weight, and SnET2 being administered to a final dose of 0.75 mg/kg body weight as described in Table 5. The time taken to administer either photosensitizer or vehicle control solution via a slow i.v. push was approximately 1 minute.

Photodynamic activation of the respective photosensitizers was undertaken 10 minutes after the completion of the administration of MRV6401, or 12 minutes after the completion of administration of SnET2. During activation, the tissue in the window chamber was exposed to the designated wavelength of filtered light from the mercury epi-illumination source. The power and total energy dose of the respective wavelengths was as described in Table 5. In addition, as described above, low power 410 nm light from the transillumination source was also used to visualize the vascular response during and after the period of activation.

Determination of Vessel Permeability Using FITC-Dextran and TRITC-Dextran

At varying time points following the photodynamic activation of MRV6401 or SnET2, a total volume of 0.1 ml of either FITC-Dextran or TRITC-Dextran (obtained from Sigma Scientific, PO Box 14508, St. Louis, Mo., 63178, USA) was administered via the i.v. tail vein route. The FITC-Dextran solutions that were used were either of molecular weight 2,000 kD (Sigma Cat. No. FD-2000s) or of molecular weight 150 kD (Sigma Cat. No. FD-150s), and the TRITC-Dextran solution was of molecular weight 155 kD (Sigma Cat. No. T1287). Prior to use, the dextrans were suspended in sterile 5% dextrose in water to a final concentration of 5% weight:volume. In all cases, a total volume of 0.1 ml of the dextrani solution was administered. The time of administration and the molecular weight of the various dextrans that were injected were as described in Table 6. In some animals the vascular permeability was determined 1-60 minutes following the completion of light irradiation using a dextran probe labeled with either FITC or TRITC, which was then followed 24 hours later by determination using a probe labeled with the other (opposite) fluorescent molecule. That is, if FITC was used immediately following irradiation, TRITC was used 24 hours later, and vice versa.

To visualize the fluorescence emitted from the FITC-Dextrans, the Leitz Ploemopak L2 cube (Cat. No. 513-420, E. Leitz Inc., Rockleigh, N.J., 07647, USA) was used. This filter cube was fitted with a BP 450-490 (450 nm-490 nm band pass) excitation filter, the RKP 510 long pass dichroic mirror and a BP 525/20 (525±10 nm) band pass barrier filter. To visualize the fluorescence emitted from the TRITC-Dextrans, the Leitz Ploemopak N2 cube (Cat. No. 513-609, E. Leitz Inc., Rockleigh, N.J., 07647, USA) was used. This filter cube was fitted with a BP 530-560 (530 nm-560 nm band pass) excitation filter, the RKP 580 long pass dichroic mirror and the LP 580 (580 nm long pass) barrier filter. The spectral characteristics of the filters in the L2 and N2 cubes were such that in animals that were injected with both FITC- and TRITC-Dextran (see Table 6), there was no fluorescence “bleed-through” from the other fluorophore. That is, when visualizing TRITC-Dextran there was no fluorescent signal from FITC-Dextran that was present in the field. Similarly, when visualizing FITC-Dextran, there was no fluorescent signal from TRITC-Dextran that was present in the field.

The quantitation of the level of either FITC-Dextran or TRITC-Dextran fluorescence in various regions of the chambers was undertaken using a minor modification of a previously described method [J. Brunner, F. Krummenaeur and H -A Lehr, “Quantification of video-taped images in microcirculation research using inexpensive imaging software” (Adobe Photoshop), Microcirculation, Vol. 7, pp 103-107, 2000]. In the studies described by Brunner et al., the imaging software utilized to quantitate the levels of fluorescence intensity in defined regions of interest within the window chamber was Adobe Photoshop. The software utilized for the studies described below was Image-Pro Plus (Media Cybernetics, 8484 Georgia Avenue, Silver Spring, Md. 20910, USA). In all other respects, the analysis undertaken below was the same as that described by Brunner et al. The level of fluorescence that was determined in both intra-vascular and extra-vascular regions using this method was directly proportional to the amount of the fluorescent labeled dextran in that region (Brunner et al., 2000).

Results and Discussion

A total of nine mice bearing dorsal window chambers were studied in these experiments, and all were evaluable for the purposes of the study. The treatment parameters utilized for each of these animals are shown in Table 5, and the fluorescent probes used to describe the post-irradiation increases in vascular permeability are shown in Table 6. The observations made during the studies are detailed in Table 7. The quantitation of the level of either FITC-Dextran or TRITC-Dextran fluorescence in various regions of the chambers was undertaken using a minor modification of a previously described method (Brunner et al., 2000) as described above. The data generated from that analysis are shown in Tables 8 and 9, and demonstrate the photodynamically enhanced delivery of molecules of varying molecular weight into the surrounding tissue.

Three animals were sensitized with SnET2, with subsequent activation by green light (530 nm-560 nm), and six animals were sensitized with MRV6401, with subsequent activation with red light (660 nm-680 nm). Control studies (not shown) demonstrated that the administration of either drug (at the doses described in Table 5) without subsequent light activation did not result in any alterations in vascular flow or leakage of any of the FITC- or TRITC-Dextran probes from the vasculature into the surrounding tissue. Similar negative results were obtained in other control studies undertaken using light irradiation alone in the absence of photosensitization with either drug (details not shown). Thus, the results described below were specific phenomena caused by the photodynamic effect on the vascular structures and surrounding tissue mediated by the combination of a photosensitizer (i.e., in this case either SnET2 or MRV6401) and activating light. Interestingly, the green wavelength band (530 nm-560 nm) utilized to activate SnET2 is a region of the spectrum where SnET2 has a low molar extinction coefficient of 4312 AU relative to the peak extinction coefficients of 165,456 at 437 nm and 52,552 at 661 nm. Thus, the effects described in Tables 7 and 8 were mediated by activation of the drug whereby its absorbance was less than 5% of its peak spectral absorbance. In the case of MRV6401, the wavelength of activation corresponded to a spectral absorbance peak for this molecule.

The data shown in Tables 5-9 describe the use of varying wavelengths of activating radiation, delivered at varying power densities for varying lengths of time, with resultant varying total energy deposition to the vessels and tissue, which can mediate the enhanced delivery of molecules from the vasculature into the tissue. The data also show that this delivery can be achieved following doses of drug and light that are sufficient to cause significant damage to the vascular structures, with accompanying loss of blood flow, or that this delivery can be achieved in selective regions with no significant loss of blood flow and no apparent long-term damage to the vasculature. The enhancement of delivery with accompanying vascular damage may be desirable in the treatment of tumors or other lesions where there would be a desire to both eradicate the diseased tissue along with delivery of a cytotoxic agent to the site. The enhancement of delivery without accompanying vascular damage may be desirable where the intention is to preserve the viability of the target tissue and vasculature, such as in the case of enhancing delivery of an antibiotic molecule to infected tissue. The selectivity of this method is particularly demonstrated by the results obtained using animals #12 and #13 in which enhanced delivery was achieved in the region of irradiation, but with maintenance of vascular integrity within all regions of the window chamber. The selective nature is critical since it allows control of the delivery to selected sites (i.e., those exposed to light), while minimizing delivery to non-irradiated sites.

The data described in Tables 8 and 9 demonstrate the enhancement of drug delivery into the target sites mediated by the photodynamic-mediated increase in vascular permeability. Results obtained in control animals irradiated in the absence of photosensitizer, or control animals administered photosensitizer but not irradiated with light, showed no increase in the level of fluorescence in the surrounding tissue. However, in the animals described in Tables 8 and 9, the level of fluorescence in the surrounding tissue, corresponding to increased levels of labeled-Dextrans, was increased as much as 6-fold 24 hours after irradiation.

The data presented in Example 3, as well as that presented in Examples 1 and 2, demonstrate that administration of a photosensitizer followed by light irradiation induces rapid changes in the vascular structures in tissues. These changes may be severe, resulting in vascular shut-down or stasis, or they may be mild, resulting in minor alteration in blood flow, with no significant long term damage to the vessels. In both cases there is a resultant permeability change in the vascular structures, which leads to localized extravasation of molecules from the blood stream into the surrounding tissue. In the specific examples described here, this induced permeability change resulted in enhanced release of macromolecules of either 150 kDalton or 2,000 kDalton molecular weight, although in principle molecules of much smaller or larger molecular weight could also be selectively released into the tissue using this method.

Therefore, this method should have broad application for the selective release of therapeutic agents with a wide range of molecular weights, such as antibiotics, chemotherapeutic agents, liposomally encapsulated agents, hormones, or diagnostic agents. While it is believed to have general application in a number of sites within an organism, this method may have particular application in mediating delivery of agents across vascular barriers that would normally limit the release of drugs from the vasculature. This is particularly applicable in the brain of many organisms where the presence of a blood brain barrier is a major limitation on the efficacy of therapies due to this barrier's capacity to exclude the release of drugs from the blood stream. That this method has been shown to increase vascular permeability while preserving the integrity of the blood vessels makes it particularly advantageous where there is a desire to limit damage to the surrounding tissue, such as in the delivery of an antibiotic to an infected wound site or the delivery of a therapeutic agent to a localized region of the brain. Alternatively, where there is a desire to both enhance the delivery of a therapeutic and at the same time achieve some degree of surrounding tissue damage, such as in the treatment of tumors, the dosimetry of this technique can be modified to achieve this result.

TABLE 1

| | | | | Dose | | | | Illumina- | |
|---|---|---|---|---|---|---|---|---|---|
| | Treat- | Sacrifice | | (mg / kg | Light | Light dose | Power density | tion time | FITC- |
| Animal # | ment date | date | Drug | b.w.) | (_) | (J / cm²) | (mW / cm²) | (sec) | Dextran |
| A34 | Jun. 14, 1999 | Jun. 18, 1999 | MV6401 | 0.05 | Blue- | 10 | 21 | 480 | No |
| | | | | | 425 nm | | | | |
| A36 | Jun. 14, 1999 | Jun. 18, 1999 | MV6401 | 0.05 | Red- | 50 (2 × 25)^{c} | 100 | 2 × 250^{c} | No |
| | | | | | 665 nm | | | | |
| A33 | Jun. 15, 1999 | Jun. 19, 1999 | EYP/D5W^{a} | 0 | Red- | 50 (2 × 25)^{c} | 100 | 2 × 250^{c} | Yes |
| | | | | | 665 nm | | | | |
| A35 | Jun. 15, 1999 | Jun. 19, 1999 | MV6401 | 0.05 | Red- | 50 (2 × 25)^{c} | 100 | 2 × 250^{c} | Yes |
| | | | | | 665 nm | | | | |
| A41 | Jul. 6, 1999 | Jul. 8, 1999 | MV6401 | 0.05 | Blue- | 50 | 80 | 625 | Yes |
| | | | | | 425 nm | | | | |
| A42 | Jul. 6, 1999 | Jul. 8, 1999 | —^{b} | 0 | — | — | — | — | — |
| A45 | Jul. 7, 1999 | Jul. 8, 1999 | EYP/D5W^{a} | 0 | Blue- | 50 | 56.6 | 883 | Yes |
| | | | | | 425 nm | | | | |
| A46 | Jul. 7, 1999 | Jul. 8, 1999 | MV6401 | 0.15 | Blue- | 50 | 56.6 | 883 | Yes |
| | | | | | 425 nm | | | | |
| A69 | Aug. 17, 1999 | Aug. 18, 1999 | SnET2 | 1.0 | Blue- | 25 | 44 | 568 | Yes |
| | | | | | 425 | | | | |
| A70 | Aug. 17, 1999 | Aug. 18, 1999 | SnET2 | 1.0 | Blue- | 50 | 44 | 1136 | Yes |
| | | | | | 425 nm | | | | |

^{a}MV6401 was formulated in Egg Yolk Phospholipid at a concentration of 0.746 / ml. Prior to administration the drug was diluted 1:50 in 5DW (5% Dextrose in Water).
^{b}Hamster A42 suffered hyperthermic burn from focused spot of mercury light source during initial calibration of instrument prior to drug administration. Power density in spot not precisely determined, however, probable power density at least 500 mW/cm².
^{c}In animals A36, A33 and A35, the light was administered in two fractions, with a 29 min refractory period between the completion of the first fraction, and the commencement of the second fraction. In all other animals the light was administered as an un-fractionated dose.

TABLE 2

| | Time of FITC- | Observations immediately | |
|---|---|---|---|
| Animal | Dextran | post FITC-Dextran | Observations 24 hr post |
| # | administration | administration | FITC-Dextran administration |
| A33 | (1) 1.5 hr post | No extravasation of | No evidence of vascular |
| | light illumination. | fluorescence evident up to | damage. No residual FITC-. |
| | (2) 24 hr post | 25 min post FITC-Dextran | Dextran could be detected by |
| | first FITC- | admin. All vessels in | fluorescence microscopy in |
| | Dextran | chamber appeared patent | the chamber following the |
| | administration | and were flowing. | administration 24 hr |
| | | | previously. A second |
| | | | administration of FITC- |
| | | | Dextran was given at this |
| | | | time, and again all vessels |
| | | | were patent with no |
| | | | extravasation of fluorescence |
| | | | evident. |
| A35 | Approximately | Extravasation of | Gross and microscopic |
| | 40 min post | fluorescence into | vascular damage. Severe |
| | PhotoPoint ™ | surrounding tissue apparent | edema apparent in tissue in |
| | therapy. | approximately 10 min post | chamber. Fluorescence |
| | | FITC-Dextran admin. | microscopy showed high |
| | | Initial poor extravasation | levels of residual extravasated |
| | | into some tissue zones | fluorescence in tissue within |
| | | probably reflective of | chamber, with no |
| | | decreased vascular flow | fluorescence apparent in |
| | | into those areas. Tissue | vasculature. All zones of |
| | | fluorescence increased for | tissue within chamber now |
| | | remainder of observation | contained residual |
| | | period (approximately 15 | fluorescence. |
| | | min.) | |
| A41 | Approximately | Results as for Animal A35 | No fluorescence microscopic |
| | 30 min post | (above). Leakage from | observations undertaken 24 hr |
| | PhotoPoint ™ | vasculature not as | post FITC-Dextran |
| | therapy. | pronounced as in A35, and | administration. Fluorescence |
| | | seemed to be better overall | microscopy was undertaken |
| | | perfusion of FITC-Dextran | 48 hr post FITC-Dextran |
| | | to all vascular structures. | administration, and showed a |
| | | | similar pattern of residual |
| | | | fluorescence to Animal A35 |
| | | | analyzed at 24 hr (above). |
| A45 | Approximately | Results as for Animal A33. | No evidence of vascular |
| | 60 min post light | No evidence of leakage of | damage. No residual FITC- |
| | illumination. | FITC-Dextran from | Dextran could be detected by |
| | | vasculature into tissue. All | fluorescence microscopy in |
| | | vessels appeared patent and | the chamber following the |
| | | were flowing. | administration 24 hr |
| | | | previously. |
| A46 | Approximately | Results as for Animal A35. | Results as for Animal A35. |
| | 50 min post | | |
| | PhotoPoint ™ | | |
| | therapy. | | |
| A69 | Approximately | Results as for Animal A35. | Results as for Animal A35. |
| | 45 min post | | |
| | PhotoPoint ™ | | |
| | therapy. | | |
| A70 | Approximately | Results as for Animal A35. | Results as for Animal A35. |
| | 60 min post | | |
| | PhotoPoint ™ | | |
| | therapy | | |

TABLE 3

| | | | | | Light dose | Power density | |
|---|---|---|---|---|---|---|---|
| | Laboratory | Chamber | | Drug Dose | (J/cm² of 664 nm | (mW/cm² of 664 | Illumination |
| Animal | Code Number | implant date | Drug | (mg/kg b.w) | light)-date | nm light) | time (sec) |
| #1 | 01-12-01 | Jan. 12, 2000 | No drug or | 0 | 0-not done | 0 | 0 |
| | | | vehicle | | | | |
| #2 | 01-10-01 | Jan. 10, 2000 | MV6401 | 0.05 | 15-Jan. 13, 2000 | 50 | 300 |
| #3 | 01-14-01 | Jan. 14, 2000 | MV6401 | 0.05 | 20-Jan. 17, 2000 | 50 | 400 |
| #4 | 01-17-04 | Jan. 17, 2000 | EYP/5DW | 0.16 ml | 20-Jan. 19, 2000 | 50 | 400 |
| | | | vehicle | | | | |

TABLE 4

| | | Observations immediately | |
|---|---|---|---|
| | Time of FITC-Dextran | post FITC-Dextran | Subsequent observations |
| Animal | administration (date) | administration | (date) |
| #1 | 8 days post chamber | No evidence of vascular or | Not done |
| | implant | tissue damage. No FITC- | |
| | (Jan. 20, 2000) | Dextran extravasation was | |
| | | evident in any parts of the | |
| | | chamber. All vessels in | |
| | | the chamber appeared | |
| | | patent and were flowing. | |
| | | No evidence of edema or | |
| | | cellular infiltrate. | |
| #2 | 4 days post chamber | No evidence of vascular or | High residual FITC- |
| | implant | issue damage in the | Dextran fluorescence |
| | 1 day post Photopoint ™ | treatment field by trans- | present in tissue within |
| | therapy | illumination or | chamber and small |
| | (Jan. 14, 2000) | fluorescence epi- | amount of fluorescence |
| | | illumination observation. | present in patent, flowing |
| | | All vessels flowing. | vessels. |
| | | Pronounced extravasation | (Jan. 15, 2000) |
| | | of FITC-Dextran from | |
| | | larger vessels in field. | |
| | | Mild edema in field. | |
| #3 | 5 days post chamber | Some evidence of focal | Pronounced focal vascular |
| | implant | vascular damage in the | damage. Some evidence |
| | 2 days post Photopoint ™ | treatment field by trans- | of tissue damage. All |
| | therapy | illumination and | zones of the chamber |
| | (Jan. 19, 2000) | fluorescence epi- | contained residual |
| | | illumination observation. | fluorescence, with |
| | | However, no obvious | evidence of high FITC |
| | | widespread tissue damage, | fluorescence in cells |
| | | however there were some | surrounding sites of |
| | | minor areas of focal | pronounced vascular |
| | | edema and cellular | damage. Fluorescence in |
| | | infiltrate. Some vessels | cells appeared to be in |
| | | flowing, some not | cytoplasm and in |
| | | flowing. Pronounced | cytoplasmic organelles, |
| | | extravasation of FITC- | with apparent exclusion |
| | | Dextran from many | from the nucleus. In these |
| | | vessels in the treatment | cells fluorescence was |
| | | field. | localized in punctate, peri- |
| | | | nuclear pattern. |
| | | | (Jan. 20, 2000) |
| #4 | 3 days post chamber | No evidence of vascular or | Not done. |
| | implant | tissue damage. No edema | |
| | 1 day post red light | or cellular infiltrate | |
| | illumination | present. No FITC- | |
| | (Jan. 20, 2000) | Dextran extravasation was | |
| | | evident in any parts of the | |
| | | chamber. All vessels in | |
| | | the chamber appeared | |
| | | patent and were flowing. | |

TABLE 5
Photosensitizer and light administration protocols for mice bearing dorsal skin window chambers.

| | Laboratory Code | Chamber implant | | Drug Dose | Wavelength of | Light dose | Power density | Illumination |
|---|---|---|---|---|---|---|---|---|
| Animal | Number | date | Drug | (mg/kg b.w) | Activating Light | J/cm² | (mW/cm² | time (sec) |
| #5 | 06-12-05 | Jun. 12, 2000 | SnET2 | 0.75 | Green | 225 | 188 | 1200 |
| | | | | | 530 nm-560 nm | | | |
| #6 | 06-26-07 | Jun. 26, 2000 | SnET2 | 0.75 | Green | 225 | 188 | 1200 |
| | | | | | 530 nm-560 nm | | | |
| #7 | 06-27-03 | Jun. 27, 2000 | SnET2 | 0.75 | Green | 225 | 188 | 1200 |
| | | | | | 530 nm-560 nm | | | |
| #8 | 12-11-11-04 | Dec. 11, 2000 | MRV6401 | 0.05 | Red | 12 | 40 | 300 |
| | | | | | 660 nm-680 nm | | | |
| #9 | 01-03-01-01 | Jan. 3, 2001 | MRV6401 | 0.05 | Red | 12 | 40 | 300 |
| | | | | | 660 nm-680 nm | | | |
| #10 | 01-03-01-03 | Jan. 3, 2001 | MRV6401 | 0.05 | Red | 12 | 40 | 300 |
| | | | | | 660 nm-680 nm | | | |
| #11 | 01-03-01-04 | Jan. 3, 2001 | MRV6401 | 0.05 | Red | 12 | 40 | 300 |
| | | | | | 660 nm-680 nm | | | |
| #12 | 01-09-01-01 | Jan. 9, 2001 | MRV6401 | 0.05 | Red | 8 | 40 | 200 |
| | | | | | 660 nm-680 nm | | | |
| #13 | 01-09-01-02 | Jan. 9, 2001 | MRV6401 | 0.05 | Red | 8 | 40 | 200 |
| | | | | | 660 nm-680 nm | | | |

TABLE 6
Administration of fluorescent Dextrans to mice bearing dorsal skin window chambers

| | | | Time of primary probe administration | Secondary probe |
|---|---|---|---|---|
| | Laboratory Code | | (post completion of light | (administered 24 hr post light |
| Animal | Number | Primary probe | irradiation) | irradiation) |
| #5 | 06-12-05 | 155 kD TRITC-Dextran | 60 min post light irradiation | No secondary probe used |
| #6 | 06-26-07 | 155 kD TRITC-Dextran | 60 min post light irradiation | No secondary probe used |
| #7 | 06-27-03 | 155 kD TRITC-Dextran | 60 min post light irradiation | No secondary probe used |
| #8 | 12-11-11-04 | 155 kD TRITC-Dextran | 10 min post light irradiation | 150 kD FITC-Dextran |
| #9 | 01-03-01-01 | 2,000 kD FITC-Dextran | 15 min post light irradiation | 155 kD TRITC-Dextran |
| #10 | 01-03-01-03 | 150 kD FITC-Dextran | 6 min post light irradiation | No secondary probe used |
| #11 | 01-03-01-04 | 2,000 kD FITC-Dextran | 19 min post light irradiation | 155 kD TRITC-Dextran |
| #12 | 01-09-01-01 | 2,000 kD FITC-Dextran | 3 min post light irradiation | 155 kD TRITC-Dextran |
| #13 | 01-09-01-02 | 155 kD TRITC-Dextran | 1 min post light irradiation | 2,000 kD FITC-Dextran |

TABLE 7
Observations made using intra-vital microscopy on mice described in Tables 5 and 6

| | Laboratory | |
|---|---|---|
| | Code | |
| Animal | Number | Observations |
| #5 | 06-12-05 | Four regions of interest were characterized. |
| | | Region 1 was in the in treatment field, with regions 2-4 being outside |
| | | treatment field. Immediately |
| | | post treatment (10 min) vascular flow was stopped in region 1, |
| | | while regions 2-4 showed significantly slower flow. |
| | | Immediately post 155 kD |
| | | TRITC-Dextran administration there was focal leakage from region 1, but no leakage from regions 2-4. Continued |
| | | observation showed no leakage |
| | | from regions 2-4. At 24 hr time point, there was no flow |
| | | in region 1, but regions 2-4 returned to pre-treatment |
| | | baseline flow. TRITC-Dextran |
| | | present in all fields, suggesting focal leakage from region |
| | | 1 had spread through entire chamber, with uptake in tissue |
| | | cells surrounding vessels. |
| #6 | 06-26-07 | Four regions of interest were characterized. Region 4 was in the in treatment field, regions 1-3 were outside |
| | | treatment field. Immediately post |
| | | treatment (10 min) vascular flow was stopped in region 4, while region 3 showed slower flow and regions 1 and 2 |
| | | showed no alteration in flow. |
| | | Twenty minutes post 155 kD TRITC-Dextran administration there was leakage from region 4, but little leakage from |
| | | regions 1-3. At 24 hr time |
| | | point, there was no flow in region 4, some very slow flow in region 3 with flow in regions 1 and 2 at pre-treatment |
| | | baseline flow. TRITC-Dextran |
| | | present in entire chamber, with cellular uptake apparent in cells in tissue surrounding blood vessels in all regions. |
| #7 | 06-27-03 | Three regions of interest were characterized. Region 3 was in the treatment field, while regions 1 and 2 were |
| | | outside the treatment field. During |
| | | treatment vascular structures in region 3 stopped flowing, while regions 1 and 2 showed some slowing, but not |
| | | stoppage, of flow. Following 155 |
| | | kD TRITC administration 1 hr after the completion of light irradiation, focal leakage was apparent from region 3, |
| | | but not regions 1 and 2. |
| | | Continued observations showed no leakage from regions 1 and 2, however at the 24 hr time point there was significant |
| | | TRITC-Dextran in the |
| | | tissues and cells within all regions of the chamber. The vessels in all regions showed significant slowing of flow |
| | | at this time point. |
| #8 | 12-11-11-04 | Three regions of interest were characterized. Region 2 was in the treatment field, while regions 1 and 3 were |
| | | outside the treatment field. During |
| | | treatment all vascular structures in all regions showed no flow alteration. Following 155 kD TRITC-Dextran |
| | | administration 10 min post light |
| | | irradiation, no significant leakage was apparent from any region up to 30 min, but some minor leakage was noted in |
| | | the treated region 2 at 1 hr. At |
| | | the 24 hr time point, there was significant leakage in region 2, with no significant fluorescence from TRITC-Dextran |
| | | present in the tissue and cells |
| | | in regions 1 and 3. There was still TRITC-Dextran present in the blood plasma in all vessels within the chamber. At |
| | | this time point, 150 kD |
| | | FITC-Dextran was administered, and the blood vessels in all regions were found to be flowing. Subsequent analysis at |
| | | 20 min post FITC-Dextran |
| | | noted leakage of FITC from vessels in the treated region 2, with some minimal leakage from the vessels in region 1, |
| | | and no leakage from the |
| | | vessels in region 3. |
| #9 | 01-03-01-01 | Three regions of interest were characterized. Region 1 was in the treatment field, with |
| | | regions 2 and 3 being outside the treatment field. During |
| | | the treatment the vessels in region 1 showed significant slowing or stoppage of flow. Administration of 2,000 kD |
| | | FITC-Dextran was undertaken 15 |
| | | min after the completion of light irradiation. This showed a significant reduction of flow in vessels in region 1, with |
| | | some minor slowing of flow |
| | | in vessels in regions 2 and 3. No leakage of 2,000 kD FITC-Dextran was apparent up to 2 hr post-administration from |
| | | any vessels in any region. |
| | | Analysis at 24 hr showed continued slowing of flow in vessels in all regions, with the presence of FITC-Dextran in |
| | | surrounding tissues and cells in |
| | | all regions. At this time point, 155 kD TRITC-Dextran was administered, and the blood vessels in all regions were |
| | | found to have slow flow, with |
| | | some minor vessels not perfused by the TRITC-Dextran |
| | | indicating up-stream blockage. Thirty minutes post TRITC-Dextran |
| | | administration there |
| | | was significant leakage from the venules in region 1, |
| | | with minimal leakage from vessels in regions 2 and 3. Subsequent |
| | | analysis at 1 hr showed |
| | | leakage from vessels in all regions. |
| #10 | 01-03-01-03 | Three regions of interest were characterized. Region 1 was in the treatment field, with |
| | | regions 2 and 3 being outside the treatment field. During |
| | | light treatment the vessels in region 1 showed significant slowing or stoppage of flow, and vessels in regions 2 and 3 |
| | | showed some minor |
| | | constriction of arterioles, but no significant alterations in flow. Administration of 150 kD FITC-Dextran was |
| | | undertaken 6 min after the completion |
| | | of light irradiation. At 1 hr post FITC-Dextran administration there was slowing of flow in vessels in all regions, |
| | | with significant leakage apparent |
| | | from the treated vessels in region 1, some minor leakage from vessels in region 3 and no leakage from vessels in region |
| | | 2. The same leakage |
| | | pattern was apparent at the 2 hr time point, and at the 24 hr time point there was FITC-Dextran present at high levels |
| | | in the tissue and cells |
| | | surrounding vessles in regions 1 and 3, with lower levels in region 2. At the 24 hr time point, it was not possible to |
| | | administer a TRITC-Dextran |
| | | probe due to damage to the tail veins in this animal. This damage was unrelated to the treatment. |
| #11 | 01-03-01-04 | Three regions of interest were characterized. Region 1 was in the treatment field, with |
| | | regions 2 and 3 being outside the treatment field. During |
| | | light treatment the vessels in region 1 showed significant slowing or stoppage of flow, while vessels in regions 2 and |
| | | 3 showed no flow alterations. |
| | | The flow alterations in region 1 resolved to normal flow at the completion of the light irradiation. Administration of |
| | | 2,000 kD FITC-Dextran was |
| | | undertaken 19 min after the completion of light irradiation, and all vessels in all fields were perfused by the |
| | | fluorescent probe. There was no |
| | | leakage from vessels in any region 10 min after FITC-Dextran administration, however at 1 hr there was some leakage |
| | | from vessels in the treated |
| | | region 1, but no leakage from vessels in regions 2 and 3. At the 24 hr time point there was significant flow reduction |
| | | in the vessels in region 1, but |
| | | no change to flow in the vessels in regions 2 and 3. Leakage of FITC-Dextran was apparent from the vessels in region 1 |
| | | with significant |
| | | FITC-Dextran uptake in tissues and cells in this region. The tissue and cells in regions 2 and 3 showed some minor |
| | | leakage, with lower tissue and |
| | | cellular levels of FITC-Dextran. |
| | | Administration of 155 kD TRITC-Dextran at this time point showed leakage from vessels |
| | | in region 1, but no |
| | | leakage from vessels in regions 2 and 3. |
| #12 | 01-09-01-01 | Three regions of interest were characterized. Region 1 was in the treatment field, with |
| | | regions 2 and 3 being outside the treatment field. During |
| | | light treatment the vessels in region 1 showed significant slowing or stoppage of flow, while vessels in regions 2 and |
| | | 3 showed no flow alterations. |
| | | Administration of 2,000 kD FITC-Dextran was undertaken 3 min after the completion of light irradiation, and all |
| | | vessels in all fields were perfused |
| | | by the fluorescent probe. There was no leakage from vessels in any region 10 min after FITC-Dextran administration, |
| | | however at 1 hr there was |
| | | leakage from vessels in the treated region 1, but no leakage from vessels in regions 2 and 3. At the 24 hr time point |
| | | there were only minor flow |
| | | alterations in vessels in region 1 and 2, and no change to flow in the vessels in region 3. Leakage of FITC-Dextran |
| | | was apparent from the vessels |
| | | in region 1 with significant FITC-Dextran uptake in tissues and cells in this region. The tissue and cells in region 2 |
| | | showed some minor leakage, |
| | | with lower tissue and cellular levels of FITC-Dextran, and there was no evidence of leakage from vessels in region 3. |
| | | Administration of 155 kD |
| | | TRITC-Dextran at this time point showed |
| | | significant on-going leakage from vessels in region 1, some minor leakage from |
| | | vessels in region 2, and |
| | | no leakage from vessels in region 3. |
| #13 | 01-09-01-02 | Three regions of interest were characterized. Region 1 was in the treatment field, with regions 2 and 3 being |
| | | outside the treatment field. During |
| | | light treatment the vessels in region 1 showed significant slowing or stoppage of flow, while vessels in regions 2 and 3 |
| | | showed no flow alterations. |
| | | Administration of 155 kD TRITC-Dextran was undertaken 1 min after the completion of light irradiation, and all vessels |
| | | in all fields were perfused |
| | | by the fluorescent probe. There was no leakage from vessels in any region 10 min and 1 hr after TRITC-Dextran |
| | | administration. At the 24 hr time |
| | | point there were flow alterations in vessels in all regions. Leakage of TRITC-Dextran was apparent from the vessels in |
| | | region 1 with significant |
| | | TRITC-Dextran uptake in tissues and cells in this region.. The tissue and cells in region 2 showed some minor leakage, |
| | | with lower tissue and |
| | | cellular levels of TRITC-Dextran, and there was no evidence of leakage from vessels in region 3. Administration of 2,000 |
| | | kD FITC-Dextran at this |
| | | time point showed significant on-going leakage from vessels in region 1, and no evidence of on-going leakage from |
| | | vessels in regions 2 and 3. |

TABLE 8
Quantitation of 155 kD TRITC-Dextran levels in
blood vessels and tissue following SnET2 mediated
photodynamic vascular permeability increase.

| Photo- | Fluorecence intensity^{a} | | | | |
|---|---|---|---|---|---|
| sensitizer | Baseline^{b} | 8 min^{b} | 20 min^{b} | 1 hr^{b} | 24 hr^{b} |
| SnET2 | Vessel | Vessel | Vessel | Vessel | Vessel |
| Animal # 6 | 28.4 ± 1.5^{c} | 105 ± 3.1 | 111 ± 2.7 | 134 ± 10 | 78 ± 1.5 |
| 06-26-07 | Tissue | Tissue | Tissue | Tissue | Tissue |
| | 30.5 ± 1.4 | 82.9 ± 1.9 | 110 ± 2.4 | 175.4 ± 3.3 | 196.4 ± 4.2 |
| SnET2 | Vessel | Vessel | Vessel | Vessel | Vessel |
| Animal # 6 | 29.9 ± 0.5 | 72 ± 1.7 | 90.6 ± 5.8 | 97.7 ± 6.2 | 55 ± 3.1 |
| 06-27-03 | Tissue | Tissue | Tissue | Tissue | Tissue |
| | 26.5 ± 0.2 | 73 ± 2.0 | 120 ± 4.4 | 127.9 ± 5.5 | 155 ± 7.1 |

^{a}As determined by adaptation of method of Brunner et al., 2000 -see Materials and Methods
^{b}Determined prior to photodynamic treatment and administration of 155 kD TRITC-Dextran Times refer to time after 155 kD TRITC-Dextran administration.
^{c}Mean ± 1 standard deviation of three readings within region of irradiation.

TABLE 9
Quantitation of 2,000 kD FITC-Dextran or 155 kD
TRITC Dextran levels in blood vessels and tissue
following MRV6401 mediated
photodynamic vascular permeability increase.

| Photosensitizer | Fluorecence intensity^{a} | | | |
|---|---|---|---|---|
| and Dextran | Baseline^{b} | 10 min^{b} | 1 hr^{b} | 24 hr^{b} |
| MRV6401 | Vessel | Vessel | Vessel | Vessel |
| Animal # 12 | 50 ± 1.6^{c} | 79.1 ± 1.95 | 83.5 ± 1.9 | 78.9 ± 2.0 |
| 01-09-01-01 | Tissue | Tissue | Tissue | Tissue |
| 2,000 kD | 50 ± 1.8 | 91.9 ± 4.4 | 121.1 ± 2.2 | 135 ± 6.6 |
| FITC-Dextran | | | | |
| MRV6401 | Vessel | Vessel | Vessel | Vessel |
| Animal # 13 | 56.6 ± 1.24 | 182.9 ± 2.6 | 168.7 ± 2.5 | 46 ± 1.4 |
| 01-09-01-02 | Tissue | Tissue | Tissue | Tissue |
| 155 kD | 59.5 ± 1.87 | 119.7 ± 1.96 | 122.9 ± 2.6 | 156 ± 1.49 |
| TRITC- | | | | |
| Dextran | | | | |

^{a}As determined by adaptation of method of Brunner et al., 2000 -see Materials and Methods
^{b}Determined prior to photodynamic treatment and administration of 2,000 kD FITC-Dextran in Animal #12 or administration of 155 kD TRITC-Dextran in Animal #13 Times refer to time after fluorescent Dextran administration.
^{c}Mean ± 1 standard deviation of three readings within region of irradiation.

The present invention is not to be limited in scope by the exemplified embodiments which are intended as illustrations of single aspects of the invention, and any photosensitizers, radiation, numerical ranges, or drugs which are functionally equivalent are within the scope of the invention. Indeed, various modifications of the invention in addition to those described herein will become apparent to those skilled in the art from the foregoing description. Such modifications are intended to fall within the scope of the appended claims. It is also to be understood that all numerical ranges given for photosensitizers, radiation, and drugs are approximate and are used solely for purposes of description.

All documents cited herein are incorporated by reference in their entirety for any purpose. The citation of any of the documents mentioned herein does not constitute an admission that the reference is prior art to the present invention.

What is claimed is:

1. A method for delivering a drug to a selected site in an organism comprising:
(a) supplying a drug to the organism;
(b) supplying a photosensitizer to a selected site in the organism; and
(c) irradiating a selected site of the organism;
wherein the intensity of said irradiation and the dose of said photosensitizer facilitate the delivery of said drug to a selected site of said organism.

2. The method of claim 1, wherein said organism is a human.

3. The method of claim 1, wherein said intensity of irradiation and dose of photosensitizer are not toxic to said organism.

4. The method of claim 1, wherein the intensity of said irradiation and the dose of said photosensitizer facilitate increased vascular permeability in a selected site in an organism without causing vascular destruction, thrombosis or vascular stasis.

5. The method of claim 1, wherein said drug is an antibiotic.

6. The method of claim 1, wherein said drug is useful in treating tumors.

7. The method of claim 1, wherein said drug is a diagnostic or reporter molecule.

8. The method of claim 1, wherein said drug is a hormone.

9. A method for increasing vascular permeability in a selected site in an organism without causing vascular destruction, thrombosis or vascular stasis comprising:
(a) supplying a photosensitizer to a selected site in the organism; and
(b) irradiating a selected site of the organism;
wherein the intensity of said irradiation and the dose of said photosensitizer facilitate increased vascular permeability in a selected site in an organism without causing vascular destruction, thrombosis or vascular stasis.

10. The method of claim 9, wherein said organism is a human.

11. The method of claim 9, wherein said intensity of irradiation and dose of photosensitizer are not toxic to said organism.
